SEQUENCE LISTING (6 sequences)

SEQ ID NO: 1 - DNA, 20 bp, Artificial Sequence
  Synthetic
ggcgttcggttttttttttc

SEQ ID NO: 2 - DNA, 22 bp, Artificial Sequence
  Synthetic
cgaataaaccgtcgtaatttcg

SEQ ID NO: 3 - DNA, 25 bp, Artificial Sequence
  Synthetic
tttgaatatttttttcggggaaatc

SEQ ID NO: 4 - DNA, 29 bp, Artificial Sequence
  Synthetic
cgaaaaaatttctattaatatcgatttcg

SEQ ID NO: 5 - DNA, 20 bp, Artificial Sequence
  Synthetic
cggagacgggatcggaagtc

SEQ ID NO: 6 - DNA, 25 bp, Artificial Sequence
  Synthetic
ttaattacgaccgactacgacaacg

(19) United States
(12) Patent Application Publication
(10) Pub. No.: US 2016/0046997 A1
(43) Pub. Date: Feb. 18, 2016

(54) BIOMARKERS FOR CERVICAL CANCER

(71) Applicant: Oslo universitetssykehus HF, Oslo (NO)

(72) Inventors: Heidi Lyng, Lommedalen (NO); Malin Lando, Oslo (NO)

(21) Appl. No.: 14/436,680

(22) PCT Filed: Oct. 17, 2013
(86) PCT No.: PCT/IB2013/003105

§ 371 Date: Apr. 17, 2015

Related U.S. Application Data
(60) Provisional application No. 61/715,475, filed on Oct. 18, 2012.

(51) Int. Cl. C12Q 1/68; G01N 33/574

(52) U.S. Cl. CPC C12Q 1/6886; G01N 33/57411; C12Q 2600/158; C12Q 2600/154; C12Q 2600/16; C12Q 2600/118

(57) ABSTRACT

The present invention relates to biomarkers for chemoradioresistant subtypes of cervical cancer. In particular the present invention relates to a method for predicting a predisposition to a chemoradioresistant cervical cancer in a subject, a method for diagnosing a chemoradioresistant cervical cancer in a subject, a method for predicting the likelihood of recurrence of cervical cancer in a cervical cancer patient under treatment, and a method for predicting the prognosis for a patient with a chemoradioresistant cervical cancer.

### FIELD OF THE INVENTION

The present invention relates to biomarkers for cervical cancer. In particular the present invention relates to biomarkers for invasive cervical cancer, kits comprising the markers, and methods of using the markers in the diagnosis and prognosis of cervical cancer.

### BACKGROUND OF THE INVENTION

Cervical cancer is one of the most common malignancies affecting women worldwide and a major cause of cancer death for women globally. Radiotherapy combined with cisplatin is the treatment of choice at the locally advanced stages. Improved therapy is needed, since more than 30% of the patients show progressive disease within 5 years after diagnosis and treatment related side effects to organs within the pelvis are frequent. Tumor stage, size, and lymph node involvement are the most powerful markers of aggressive disease, but do not fully account for the observed variability in outcome and are not biologically founded.

A better handling of the disease may be provided by the discovery of efficient biomarkers for therapeutic planning and intervention, but requires more insight into the mechanisms underlying cervical carcinogenesis and treatment relapse.

During carcinogenesis, genetic and epigenetic alterations drive the evolution of tumor towards increased malignancy and treatment resistance. The changes enable tumor cells to overcome microenvironmental constraints, sustain proliferation, and invade adjacent tissues and distinct organs. Gene dosage alterations like gains and losses regulate the expression of genes and are motive forces for this evolution.

Tumor cells bearing an increasing number of gains and losses successively emerge and are selected for based on the growth advantage caused by the genetic changes. Discovery and functional assessment of gene dosage alterations involved in carcinogenesis are therefore essential for understanding the biology of the disease.

At the locally advanced stages of cervical cancer, numerous gene dosage alterations and severe aneuploidy are frequently seen. Moreover, pronounced intratumor heterogeneity in the gains and losses exists within the tumors, reflecting a high genetic instability.

The consequences of these alterations for the tumor phenotype are difficult to predict, since large chromosomal regions involving multiple genes are generally affected and some aberrations may be random events without biological significance. Genome wide screening of DNA copy numbers in a decent number of patients enables identification of recurrent gene dosage alterations; i.e., alterations characteristic of the disease, and alterations associated with the clinical outcome, which are likely to be important in carcinogenesis and treatment resistance.

Combining the data with expression profiles of the same tumors reveals the genes that are regulated primarily by the genetic events. The potential of this integrative strategy was recently demonstrated in a study on 15 early stage cervical cancers, where genes affected by aberrations on 1q, 3q, 11q, and 20q were reported.

Genetic events promoting tumor evolution and treatment resistance have, however, not been explored on a genome wide scale, and their biological meaning has not been addressed.

### SUMMARY OF THE INVENTION

The present invention relates to biomarkers for cervical cancer. In particular the present invention relates to biomarkers for invasive cervical cancer, kits comprising the markers, and methods of using the markers in the diagnosis and prognosis of cervical cancer.

For example, in some embodiments, the present invention provides a kit for detecting loss of gene expression associated with cervical cancer, comprises (e.g., consisting essentially of): a) a first gene expression informative reagent for identification of loss or decrease of gene expression of a first gene located at the chromosomal region 3p11.2-p14.2; and b) a second gene expression informative reagent for identification of loss or decrease of gene expression of a second gene located at the chromosomal region 3p11.2-p14.2, as well as methods and uses of the kits and reagents for diagnosing and providing a prognosis for cervical cancer (e.g., aggressive cervical cancer). In some embodiments, the kits and methods comprise additional gene expression informative reagents for identification of loss or decrease in gene expression in one or more additional genes. In some embodiments, the genes are, for example, THOC7, PSMD6, SLC25A26, TMF1, RYBP, SHQ1, EBLN2, or GBE1. In some embodiments, the reagents are, for example, nucleotide probes that specifically bind to the genes, antibodies that specifically bind to polypeptides encoded by the genes, first and second pairs of primers for amplifying the first and second genes, or sequence primers for sequencing said first and second genes. In some embodiments, loss or expression is detected in a sample from a subject (e.g., a tissue sample, a cell sample, or a blood sample). In some embodiments, computer implemented methods are utilized to determine loss or decrease in expression (e.g., to analyze variant information and display the information to a user).

In some embodiments, the present invention provides the step of treating subjects identified as having cervical cancer (e.g., invasive cervical cancer) using the methods described herein. In some embodiments, the treatment results in decrease in at least one symptom or measure of aggressiveness of the cervical cancer.

In some embodiments, the reagents, kits, and methods described herein are used to provide a prognosis to a subject (e.g., decreased or increased survival from cervical), for example, based on the level of invasiveness of the cervical cancer.

Additional embodiments are described herein.

### BRIEF DESCRIPTION OF THE FIGURES

FIG. 1. Genetic loss on chromosome 3p for intraepithelial lesions and invasive carcinomas of the uterine cervix. (A) Frequency of loss for intraepithelial lesions (CIN2/3), intraepithelial lesions adjacent to invasive carcinoma (SCC-CIN3), and invasive carcinomas at different stages. (B) 3p gene dosage profile of 92 invasive carcinomas. (C) Frequency of loss for the invasive stages in (A) combined. (D) P values in univariate Cox regression analysis of locoregional control (LC) and progression free survival (PFS) for the patients in (C), showing the correlation between gene dosage and clinical outcome along 3p.

FIG. 2. Downregulated genes within 3p11.2-p14.2 in carcinomas with 3p loss. Correlation coefficients from Spearman's rank correlation analysis of gene dosage against expression in 77 invasive carcinomas, including all genes within the 3p11.2-p14.2 region.

FIG. 3. Protein expression of the candidate targets RYBP and TMF1. (A) Immunohistochemical staining of RYBP (upper) and TMF1 (lower) in normal cervix and invasive cervical carcinoma with high and low protein expression. (B) RYBP and TMF1 protein expression of 86 invasive carcinomas with no loss (NL, gene dosage>0.9, n=35), moderate loss (ML, 0.5<gene dosage≦0.9, n=27), or severe loss (SL, gene dosage≦0.5, n=24) of the RYBP and TMF1 gene, respectively.

FIG. 4. Network and validation of genes associated with 3p12-p14 loss. (A) Second degree protein interaction network of eight candidate 3p target genes, showing the direct interaction partners of the candidates and of their direct partners. (B) Gene expressions by cDNA microarrays of 90 invasive carcinomas with no loss (gene dosage>0.9; n=37) or loss (gene dosage≦0.9; n=53) of 3p12-p14 for selected genes in the network.

FIG. 5. Prognostic impact of the candidate target genes. (A) Unsupervised hierarchical clustering of 77 invasive carcinomas based on the expression of the eight candidate 3p target genes. (B) Kaplan-Meier curves of locoregional control and progression free survival for the patients in the two clusters identified in (A). (C) 3p target gene score of 77 invasive carcinomas with no loss (NL, gene dosage>0.9, n=35), moderate loss (ML, 0.5<gene dosage≦0.9, n=27), or severe loss (SL, gene dosage≦0.5, n=24) of 3p12-p14. (D) Kaplan-Meier curves of progression free survival for the 77 patients in (A) with high and low 3p target gene score. (E) Kaplan-Meier curves of progression free survival for patients in the validation cohort with high and low 3p target gene score. (B, D, E) P-values in log rank test and number of patients are indicated. (D, E) The number of patients in each group was chosen to achieve the largest difference in survival between the groups.

FIG. 6. Clinical outcome for patients with or without recurrent 3p loss. Kaplan-Meier curves of locoregional control and progression free survival for 92 cervical cancer patients with no loss (NL), moderate loss (ML), and severe loss (SL) of 3p11 2 p14 2.

FIG. 7. Gene expression of the candidate target genes in carcinomas with or without recurrent 3p loss. A total of 77 cervical tumors are divided into two groups based on the aCGH data of the most significant aCGH probe in the Cox regression analysis (70.7 Mb); Loss, n=46; No loss, n=31. P-values from Student's t-test or Mann-Whitney U test are indicated.

FIG. 8. Immunohistochemical staining and Western blot of RYBP in siRNA transfected SiHa cells.

FIG. 9. siRNA knockdown of RYBP, TMF1, and PSMD6 in cervical cancer cell lines. (A) Western blot of HeLa, SiHa, and CaSki cells showing RYBP and TMF1 protein expression in control cells transfected with siGENOME Non-Targeting siRNA and cells transfected with RYBP or TMF1 siGENOME SMARTpool. (B) Flow cytometry histogram of SiHa cells stained with Hoechst 33258, showing number of cells versus DNA content in control cells transfected with siGENOME Non-Targeting siRNA and cells transfected with RYBP, TMF1, and PSMD6 siGENOME SMARTpool. (C) Change in gene expression of the 8 candidate target genes in HeLa, SiHa, and CaSki cells transfected with RYBP, TMF1, and PSMD6 siGENOME SMARTpool compared to control cells transfected with siGENOME Non-Targeting siRNA.

FIG. 10. Clinical outcome for patients with high or low expression of the candidate target genes. Kaplan-Meier curves for progression free survival of cervical cancer patients with high and low gene expression of the 3p target genes.

FIG. 11. Methylation-specific PCR (MSP) of RYBP, TMF1, and PSMD6. (A) MSP products for CpGenome Universal Methylated DNA (IVD), unmodified DNA (UD), water, and a selected tumor with detected methylation for PSMD6 (P-210). (B) MSP data on 70 tumors with and without 3p loss.

### DEFINITIONS

Prior to discussing the present invention in further details, the following terms and conventions will first be defined:

As used herein, the term “sample” relates to any liquid or solid sample collected from an individual to be analyzed. In one embodiment, the sample is liquefied at the time of assaying. In another embodiment, the sample is suspension of single cells disintegrated from a tissue biopsy such as a tumor biopsy. In some embodiments, the sample is a tissue sample, for example, a tissue section mounted on a slide. In some embodiments, the sample comprises genomic DNA, mRNA or rRNA. In another embodiment of the present invention, a minimum of handling steps of the sample is necessary before measuring the expression of a RNA/cDNA. In the present context, the subject “handling steps” relates to any kind of pre-treatment of the liquid sample before or after it has been applied to the assay, kit or method. Pre-treatment procedures includes separation, filtration, dilution, distillation, concentration, inactivation of interfering compounds, centrifugation, heating, fixation, addition of reagents, or chemical treatment. In accordance with the present invention, the sample to be analyzed is collected from any kind of mammal, including a human being, a pet animal, and a zoo animal. In yet another embodiment of the present invention, the sample is derived from any source such as body fluids. Preferably, this source is selected from the group consisting of milk, semen, blood, serum, plasma, saliva, faeces, urine, sweat, ocular lens fluid, cerebral spinal fluid, cerebrospinal fluid, ascites fluid, mucous fluid, synovial fluid, peritoneal fluid, vaginal discharge, vaginal secretion, cervical discharge, cervical or vaginal swab material or pleural, amniotic fluid and other secreted fluids, substances, cultured cells, and tissue biopsies. One embodiment of the present invention relates to a method according to the present invention, wherein said body sample or biological sample is selected from the group consisting of blood, vaginal washings, cervical washings, cultured cells, tissue biopsies such as cervical biopsies, and follicular fluid. Another embodiment of the present invention relates to a method according to the present invention, wherein said biological sample is selected from the group consisting of blood, plasma and serum. The sample taken may be dried for transport and future analysis. Thus the method of the present invention includes the analysis of both liquid and dried samples.

As used herein, the term “chromosome region” refers to a portion of a chromosome. Several chromosome regions have been defined by convenience in order refer to the location of genes, for example the distinction between chromosome region p and chromosome region q. In diploid organisms, homologous chromosomes get attached to each other by the centromere. The centromere divides each chromosome into two regions: the smaller one, which is the p region, and the bigger one, the q region. At either end of a chromosome is a telomere, and the areas of the p and q regions close to the telomeres are the subtelomeres, or subtelomeric regions. The areas closer to the centromere are the pericentronomic regions. Finally, the interstitial regions are the parts of the p and q regions that are close to neither the centromere nor the telomeres, but are roughly in the middle of p or q. The chromosomal region may be further defined by reference to the conventional banding pattern of the chromosome. For example, 3p11.2 refers to chromosome 3, p arm, with the numbers that follow the letter representing the position on the arm: band 1, section 1, sub-band 2. The bands are visible under a microscope when the chromosome is suitably stained. Each of the bands is numbered, beginning with 1 for the band nearest the centromere. Sub-bands and sub-sub-bands are visible at higher resolution. As a further example, 3p11.2-p14.1, refers to the region on the p arm of chromosome 3 from band 1, section 1, sub-band 2 to band 1, section 4, sub-band 1.

The term “dosage” as used herein refers to the number of copies of a chromosomal region, or portion thereof, or a gene present in a cell or nucleus. Thus, the “chromosomal region dosage” is the number of copies of a particular chromosomal region, or portion thereof, in a cell or nucleus. Likewise, the “gene dosage” is the number of copies of a particular gene in a cell or nucleus.

The genes described herein are identified by the following gene accession numbers:

| Gene Symbol | UGRepAcc |
|---|---|
| THOC7 | NM_025075.2 |
| PSMD6 | NM_014814.1 |
| SLC25A26 | JF432619.1 |
| TMF1 | NM_007114.2 |
| RYBP | NM_012234.5 |
| SHQ1 | NM_018130.2 |
| EBLN2 | NM_018029.3 |
| GBE1 | NM_000158.3 |

The term “cervical cancer” as used herein refers to a malignant neoplasm of the cervix uteri or cervical area. A typical treatment consists of surgery (including local excision) in early stages and chemotherapy and radiotherapy in advanced stages of the disease. Following chemotherapy and radiotherapy, the cervical cancer may relapse as a subtype of cervical cancer resistant to the at least one of the presently available chemotherapies or radiotherapies.

### DETAILED DESCRIPTION OF THE INVENTION

The present invention relates to biomarkers for cervical cancer. In particular the present invention relates to biomarkers for invasive cervical cancer, kits comprising the markers, and methods of using the markers in the diagnosis and prognosis of cervical cancer.

Although the p12-p14 region constitutes the less gene rich part of chromosome 3p, the target genes of the loss and its role in the pathogenesis of cervical cancer remain to be clarified. The breakpoint of the p14 band is close to the fragile region FRA3B at p14.2 (Kok et al., Adv Cancer Res 1997; 71:27-92). The FHIT gene, encompassing the fragile region, and the more centromeric genes FOXP1, RYBP, and SHQ1 at 3p13, ROBO1 at 3p12.3, and GBE1 at 3p12.2, have been found to be frequently deleted and downregulated in carcinomas of the prostate, breast, lung, and ovary and proposed targets of 3p losses (Birch et al., Mol Carcinog 2008; 47:56-65; Taylor et al., Cancer Cell 2010; 18:11-22; Zabarovsky et al., Oncogene 2002; 21:6915-6935). In cervical cancer, RYBP and GBE1 have been reported to be highly downregulated in tumors with 3p loss (Lando et al., PLoS Genet 2009; 5:e1000719), in line with these findings.

The present disclosure investigated the recurrent 3p12-p14 loss of cervical squamous cell carcinomas (SCC) in the timeline of the neoplastic progression and identify candidate target genes of the loss. DNA copy number alterations on 3p were compared across 49 precancerous and 92 cancerous lesions to find the onset of the genetic event. To depict candidate target genes. An integrative copy number and expression analysis of 77 tumors, where all genes within the recurrent 3p region were included, and selected the genes that were highly downregulated in cases with 3p loss, was performed. The selected genes were further subjected to a combined global network and gene ontology (GO) analysis based on the expression profiles, to investigate whether their downregulation was consistent with the activation of tumorigenic pathways. Finally, it was explored whether transcriptional downregulation of the candidate genes was associated with poor clinical outcome, as reported for the recurrent 3p loss (Lando et al., supra). The feasibility of this approach was demonstrated by the identification of eight candidate 3p target genes with prognostic impact in an independent cohort of 74 cervical cancer patients.

A complete transcript mapping of the recurrent 3p loss in cervical squamous cell was performed and eight candidate target genes of the loss were identified. The study is the first to include all known genes in the search for targets of this event, resulting in several novel candidates. Their function as 3p targets was supported, and increased knowledge of their role in carcinogenesis was obtained by global pathway analysis and survival analysis in different patient cohorts. Moreover, comparison of 3p copy numbers across intraepithelial and invasive lesions identified the time point during disease progression, when the loss constitutes a significant selection advantage. Loss of 3p12-p14 plays an important role in the pathogenesis of cervical cancer and is valuable to implement in the clinical decision-making.

Loss of 3p12-p14 was rare (2%) in the high-grade precancerous lesions compared to the findings in invasive carcinomas or to alterations on chromosomes 1, 3q, and 20, which are rather frequent in high-grade CIN lesions (Wilting et al., Cancer Res 2009; 69:647-655). Studies using 3p14.2 (Wistuba et al., Cancer Res 1997; 57:3154-3158) and 3p13 (Dasgupta et al., Mol Pathol 2003; 56:263-269) DNA markers for loss of heterozygosity analysis, have shown similar results. The loss is therefore not important for the development of highgrade CIN, but rather for invasive growth. The loss on 3p was more common in CIN3 adjacent to invasive carcinoma than in CIN2/3 lesions, and has been found at a high frequency (75%) in a small study on microinvasive carcinomas (Wistuba et al. supra). At invasive stages, the intratumor heterogeneity of this loss has been reported to be low compared to that of many other chromosomal alterations (Lando et al., supra; Lyng et al., Int J Cancer 2004; 111:358-366). The 3p12-p14 loss is thus involved in the acquisition of invasiveness, or during the invasive phase. In both cases, this constitutes a selection advantage towards a more aggressive disease and a treatment resistant tumor phenotype.

Eight genes within the 3p12-p14 region were strongly downregulated in the invasive carcinomas with loss, as was confirmed at the protein level for RYBP and TMF1, and they were therefore identified as target genes of the loss. GBE1 has been described as a target of the 3p loss in ovarian cancer (Birch et al., Mol Carcinog 2008; 47:56-65), and SHQ1 and RYBP as targets in prostate cancer (Taylor et al., Cancer Cell 2010; 18:11-22).

A function of the candidate genes as 3p targets indicates that their loss promotes the activation of tumorigenic pathways. This was supported from the combined network and GO analysis, which also depicted biological processes that might be involved. Moreover, the differential expression of selected interaction partners in the network, CTNNB1, TUSC2, CUL4A, and TFDP1, was confirmed in a cDNA microarray data set. A connection between RYBP repression and upregulation of E2F4 and its dimerization partner TFDP1 was seen in the network. Upregulation of E2F4 and TFDP1 can promote cell cycle progression in HPV infected tumors (Adams and Kaelin, Semin Cancer Biol 1995; 6:99-108), and loss of RYBP may contribute to increased proliferation through this mechanism. RYBP is also a pro-apoptotic gene (Zheng et al., J Biol Chem 2001; 276:31945-31952), and its loss may explain the observed downregulation of several pro-apoptotic genes, including a number of caspases, APAF1, and DIABLO. APAF1 downregulation and a suppressed apoptosis may also be linked to the loss of EBLN2 through its connection to TUSC2 (Ji and Roth, J Thorac Oncol 2008; 3:327-330). Moreover, TMF1 can attenuate tumor progression and induce apoptosis under nutrient deprived conditions Zheng et al., supra), indicating that TMF1 loss may promote increased proliferation and apoptosis resistance. The network data therefore described an association between loss of RYBP, TMF1, and EBLN2 and an aggressive tumor phenotype characterized by increased proliferation and apoptosis resistance.

PSMD6 encodes a subunit of the constitutive proteasome, which can switch to the inducible immunoproteasome upon cytokine stimulation (Frankland-Searby and Bhaumik, Biochim Biophys Acta 2012; 1825:64-76). Loss of PSMD6 and downregulation of its interaction partner encoded by PSMD5 may impair this switch and reduce the tumor immune response. The SHQ1 encoded protein has been shown to attenuate growth of prostate cancer (Nallar et al., PLoS One 2011; 6:e24082), THOC7 is involved in nuclear export of transcripts, including viral mRNA (Nallar et al., supra; E1 et al., FEBS Lett 2009; 583:13-18), SLC25A26 encodes a mitochondrial transport protein (Nallar et al., supra; del Arco et al., J Biol Chem 2004; 279:24701-24713, and GBE1 participates in energy metabolism. SHQ1 showed a connection to CTNNB1 through CTNND1. The repression of CTNNB1 may indicate disrupted stability and integrity of the CDH1-CTNNB1 complex, and thereby increase proliferation, migration, and invasion (Tian et al., J Biomed Biotechnol 2011; 2011:567305). Several stress genes known to be key regulators of DNA repair and maintenance of genome integrity, like FANCD2, MLH1, and PLK3, were downregulated, indicating impaired DNA damage response and genomic instability (Bogliolo et al., Mutagenesis 2002; 17:529-538; Loffler et al., Exp Cell Res 2006; 312:2633-2640; Vilar and Gruber, Nat Rev Clin Oncol 2010; 7:153-162). Upregulation of CUL4A and downregulation of XPC may also indicate a restricted DNA damage response (Jackson and Xiong, Trends Biochem Sci 2009; 34:562-570), although CUL4Aay plays a role in apoptosis, cell cycle control, and proliferation as well. Altogether, the network and GO analyses support a tumor suppressor role of the candidate genes and hence a function as 3p targets. Moreover, the results serve as a basis for deciphering the molecular mechanisms underlying the selection advantage of the 3p12-p14 loss in carcinogenesis.

A target gene function of the candidates, and their importance in disease progression, were further supported by the prognostic impact of the target gene signature, and the confirmation of this impact in an independent cohort of patients. In particular, the prognostic impact was independent of existing clinical markers, indicating that the candidate genes provide a valuable supplement to these markers for the selection of patients with a high probability of locoregional and distant relapse after chemoradiotherapy. In accordance with this result, the loss of the 3p12-14 region emerged as an independent prognostic factor in previous work (Lando et al, supra). In the present study, it was shown that the prognostic impact of the genetic event can be covered by the loss of eight specific genes. These genetic markers are of value for assessing aggressiveness already at early invasive stages, when the loss is selected for in carcinogenesis.

In some embodiments, the dosage or expression level of at least one gene in the 3p12-p14 level is measured in order to provide a diagnosis or prognosis of cervical cancer. Examples of genes include, but are not limited to, THOC7, PSMD6, SLC25A26, TMF1, RYBP, SHQ1, EBLN2, or GBE1.

Determination of the dosage of the foregoing chromosomal regions and genes provides important information for making a number of different clinical diagnoses, prognoses, and predictions. In some embodiments, the alteration in dosage, preferably a decrease in dosage or expression is indicative of cancers that are likely to or have become invasive. In some embodiments, the alteration in gene dosage indicates the likelihood of recurrence of cervical cancer. In some embodiments, the alteration in gene dosage or expression indicates poor survival of a patient. In some embodiments, the alteration in gene dosage indicates a prognosis for 60 months survival or more than 60 months survival (e.g., more than 10 year survival or 15 year survival). In another embodiment of the present invention, the prognosis for less than 60 months, such as less than 36 months, such as less than 24 months of survival. In some embodiments, the alteration in gene dosage indicates that the patient is a candidate for treatment with a particular therapy or therapeutic agent. In some embodiments, the alteration in gene dosage indicates the efficacy (e.g., a poor efficacy) of a treatment of a subtype of cervical cancer in a subject.

In some embodiments, the methods of the present disclosure comprise determining the gene dosage are combined with further determination of expression level(s) of selected genes, which correlate with the diagnosis, prognosis, or agressivness of cervical cancer.

1. Diagnostic Applications

The present invention provides DNA, RNA and protein based diagnostic methods that either directly or indirectly detect the dosages and/or gene expression levels as described above. The present invention also provides compositions and kits for diagnostic purposes.

The diagnostic methods of embodiments of the present invention may be qualitative or quantitative. Quantitative diagnostic methods may be used, for example, to discriminate via a cut-off or threshold level. Where applicable, qualitative or quantitative diagnostic methods may also include amplification of target, signal or intermediary (e.g., a universal primer). An initial assay may confirm the presence of a change in gene dosage, but not identify the specific gene. A secondary assay is then performed to determine the identity of the particular gene in which dosage is changed, if desired. The second assay may use a different detection technology than the initial assay.

The dosage of chromosomal regions and/or genes, as well as expression of the genes of embodiments of the present invention, may be detected along with other markers in a multiplex or panel format. Markers are selected for their predictive value alone or in combination with the identified chromosomal regions and/or genes. Markers for other cancers, diseases, infections, and metabolic conditions are also contemplated for inclusion in a multiplex of panel format.

Any patient sample suspected of containing the cancer markers may be tested according to the methods of embodiments of the present invention. By way of non-limiting examples, the sample may be tissue (e.g., a cervical biopsy sample), blood, urine, cervical/vaginal secretions or a fraction thereof (e.g., plasma, serum, urine supernatant, urine cell pellet or cervical cells).

The dosage of chromosomal regions and/or genes of embodiments of the present invention, as well expression of the genes, may be detected using a variety of nucleic acid techniques known to those of ordinary skill in the art, including but not limited to: nucleic acid sequencing; nucleic acid hybridization; and, nucleic acid amplification.

Illustrative non-limiting examples of nucleic acid sequencing techniques include, but are not limited to, chain terminator (Sanger) sequencing and dye terminator sequencing. Those of ordinary skill in the art will recognize that because RNA is less stable in the cell and more prone to nuclease attack experimentally RNA is usually reverse transcribed to DNA before sequencing.

Chain terminator sequencing uses sequence-specific termination of a DNA synthesis reaction using modified nucleotide substrates. Extension is initiated at a specific site on the template DNA by using a short radioactive, or other labelled, oligonucleotide primer complementary to the template at that region. The oligonucleotide primer is extended using a DNA polymerase, standard four deoxynucleotide bases, and a low concentration of one chain terminating nucleotide, most commonly a di-deoxynucleotide. This reaction is repeated in four separate tubes with each of the bases taking turns as the di-deoxynucleotide. Limited incorporation of the chain terminating nucleotide by the DNA polymerase results in a series of related DNA fragments that are terminated only at positions where that particular di-deoxynucleotide is used. For each reaction tube, the fragments are size-separated by electrophoresis in a slab polyacrylamide gel or a capillary tube filled with a viscous polymer. The sequence is determined by reading which lane produces a visualized mark from the labelled primer as you scan from the top of the gel to the bottom.

Dye terminator sequencing alternatively labels the terminators. Complete sequencing can be performed in a single reaction by labelling each of the di-deoxynucleotide chain-terminators with a separate fluorescent dye, which fluoresces at a different wavelength.

Illustrative non-limiting examples of nucleic acid hybridization techniques include, but are not limited to, in situ hybridization (ISH), microarray, and Southern or Northern blot.

In situ hybridization (ISH) is a type of hybridization that uses a labelled complementary DNA or RNA strand as a probe to localize a specific DNA or RNA sequence in a portion or section of tissue (in situ), or, if the tissue is small enough, the entire tissue (whole mount ISH). Examples of suitable ISH methods include, but are not limited to, fluorescence in situ hybridization (FISH), colorimetric in situ hybridization (CISH) or silver in situ hybridization (SISH). DNA ISH can be used to determine the structure of chromosomes. RNA ISH is used to measure and localize mRNAs and other transcripts within tissue sections or whole mounts. Sample cells and tissues are usually treated to fix the target transcripts in place and to increase access of the probe. The probe hybridizes to the target sequence at elevated temperature, and then the excess probe is washed away. The probe that was labelled with either radio-, fluorescent- or antigen-labelled bases is localized and quantitated in the tissue using either autoradiography, fluorescence microscopy or immunohistochemistry, respectively. ISH can also use two or more probes, labelled with radioactivity or the other non-radioactive labels, to simultaneously detect two or more transcripts.

In some embodiments, the dosage of the chromosomal regions and/or genes is detected using FISH, CISH or SISH. Nucleic acid probes specific for the region or gene are labelled with appropriate fluorescent or other markers and then used in hybridizations. The Examples section provided herein sets forth one particular protocol that is effective for measuring deletions but one of skill in the art will recognize that many variations of this assay can be used equally well. Specific protocols are well known in the art and can be readily adapted for use. Guidance regarding methodology may be obtained from many references including: In situ Hybridization: Medical Applications (eds. G. R. Coulton and J. de Belleroche), Kluwer Academic Publishers, Boston (1992); In situ Hybridization: In Neurobiology; Advances in Methodology (eds. J. H. Eberwine, K. L. Valentino, and J. D. Barchas), Oxford University Press Inc., England (1994); In situ Hybridization: A Practical Approach (ed. D. G. Wilkinson), Oxford University Press Inc., England (1992)); Kuo, et al., Am. J. Hum. Genet. 49:112-119 (1991); Klinger, et al., Am. J. Hum. Genet. 51:55-65 (1992); and Ward, et al., Am. J. Hum. Genet. 52:854-865 (1993)). There are also kits that are commercially available and that provide protocols for performing FISH assays (available from e.g., Oncor, Inc., Gaithersburg, Md.). Patents providing guidance on methodology include U.S. Pat. Nos. 5,225,326; 5,545,524; 6,121,489 and 6,573,043. All of these references are hereby incorporated by reference in their entirety and may be used along with similar references in the art and with the information provided in the Examples section herein to establish procedural steps convenient for a particular laboratory.

In some embodiments, the dosage of the chromosomal region and/or gene is determined by a microarray based method. Different kinds of biological assays are called microarrays including, but not limited to: DNA microarrays (e.g., cDNA microarrays and oligonucleotide microarrays); protein microarrays; tissue microarrays; transfection or cell microarrays; chemical compound microarrays; and, antibody microarrays. A DNA microarray, commonly known as gene chip, DNA chip, or biochip, is a collection of microscopic DNA spots attached to a solid surface (e.g., glass, plastic or silicon chip) forming an array for the purpose of expression profiling or monitoring expression levels for thousands of genes simultaneously. The affixed DNA segments are known as probes, thousands of which can be used in a single DNA microarray. Microarrays can be used to identify disease genes by comparing gene expression in disease and normal cells. Microarrays can be fabricated using a variety of technologies, including but not limiting: printing with fine-pointed pins onto glass slides; photolithography using pre-made masks; photolithography using dynamic micromirror devices; ink jet printing; or, electrochemistry on microelectrode arrays.

In some embodiments, absolute tumor DNA copy numbers is determined by GeneCount, a method for genome-wide calculation of absolute copy numbers from clinical array comparative genomic hybridization data. The tumor cell fraction is reliably estimated in the model. Data consistent with FISH results are achieved. Array comparative genomic hybridization (aCGH) is widely used for genome-wide mapping of DNA copy number changes in malignant cells. Genetic gains and losses impact gene expression levels, and thereby promote tumor growth and progression.

In some embodiments of the present invention, the gene dosage is determined by array comparative genomic hybridization (aCGH). The relative values achieved in aCGH experiments are influenced by the total DNA content (ploidy) of the tumor cells, the proportion of normal cells in the sample, and the experimental bias, in addition to the DNA copy numbers. In another embodiment of the present invention, the gene dosage is the ratio of absolute DNA copy number in said chromosomal regions and the DNA ploidy of the sample. In another embodiment of the present invention, the proportion of normal cells in the sample is estimated and DNA ploidy of the sample is corrected for the presence of normal cells in the sample. In the present context, ploidy refers to the number of complete sets of chromosomes in a biological cell. In humans, the somatic cells that compose the body are diploid (containing two complete sets of chromosomes, one set derived from each parent), but sex cells (sperm and egg) are haploid. In contrast, tetraploidy (four sets of chromosomes) is a type of polyploidy and is common in plants, and not uncommon in amphibians, reptiles, and various species of insects. The number of chromosomes in a single non-homologous set is called the monoploid number (x). The haploid number (n) is the number of chromosomes in a gamete of an individual. Both of these numbers apply to every cell of a given organism. For humans, x=n=23; a diploid human cell contains 46 chromosomes: 2 complete haploid sets, or 23 homologous chromosome pairs. The values are presented as intensity ratios between tumor and normal DNA. The data are normalized so that the ratio of 1.0 is the baseline for the analysis, and corresponds to two DNA copies in near diploid (2n) tumors.

In some preferred embodiments, the copy number changes are identified from the ratios deviating from the baseline, using statistical methods for ratio smoothing and breakpoint detection. To assign an absolute copy number to each ratio level identified by the statistical analysis and thereby score genetic aberrations are, however, challenging. In aneuploid tumors with gross alterations in the DNA content, the baseline represents a copy number other than 2, like 3 or 4 in tri- or tetraploid tumors, or a non-integer value when the DNA content differs from n, 2n, 3n, . . . mn. The presence of normal cells within the sample and experimental bias reduce the ratio dynamics. In the present context, euploidy refers to the state of a cell or organism having an integral multiple of the monoploid number, possibly excluding the sex-determining chromosomes. For example, a human cell has 46 chromosomes, which is an integer multiple of the monoploid number, 23. A human with abnormal, but integral, multiples of this full set (e.g. 69 chromosomes) would also be considered as euploid. Aneuploidy is the state of not having euploidy. Moreover, in many tumors, several subpopulations of malignant cells with different genetic characteristics exist, leading to intratumor heterogeneity in the DNA copy numbers and increased complexity in the data. Unreliable results occur, therefore, when common ratio levels are used to score gains and losses in tumors with different ploidy and normal cell content. The confounding effect caused by normal cells within tumor samples is recognized as a problem in aCGH analyses and has been handled by excluding low purity samples or correcting the ratio levels based on histological examination of tumor sections. The latter approach is not satisfactory because only the proportion of connective tissue surrounding the tumor parenchyma, and not the infiltrating immune cells, is precisely quantified. Moreover, the measurements cannot be performed on exactly the same tissue as used in the aCGH experiment and may, therefore, not be representative.

In preferred embodiments utilizing GeneCount, the proportion of normal cells in the sample is estimated and corrected for and possible intratumor heterogeneity in DNA copy numbers is considered. Inputs to the model are the DNA index (DI, where DI=1/2·tumor ploidy), tumor cell fraction, experimental bias, and aCGH ratios. Predetermined measures of tumor ploidy, determined either by flow or image based cytometry, are useful. The tumor cell fraction can be determined by, for example, flow cytometry on the same part of the sample as used in the aCGH experiment. In cases of unknown normal cell content, the tumor cell fraction is estimated in the model. The experimental bias is determined from the X-chromosome ratio in aCGH experiments where male and female DNA is compared. Smoothed ratio levels from any existing statistical analysis tools for breakpoint detection can be used. The principle of GeneCount is outlined in detail in Lyng et al. (2008).

Current methods for analysis of aCGH data generally score genetic gains and losses based on ratio levels. The breakpoints in individual tumors can be detected with high accuracy by use of statistical algorithms like GLAD and CGH-Explorer. However, the existing downstream analyses, using common ratio levels for scoring aberrations across tumors, fail to identify gains and losses in cases of high ploidy and normal cell content. By the use of GeneCount, the ratio levels are replaced with the absolute copy numbers relative to the total DNA content as measures of gene dosage, which can be compared across tumors regardless of ploidy and normal cell content. The absolute DNA copy number relative to the total DNA content, or gene dosage, is comparable also across tumors.

In some embodiments, the dosage of the chromosomal region and/or gene is determined by Northern or Southern blotting. Southern and Northern blotting is used to detect specific DNA or RNA sequences, respectively. DNA or RNA extracted from a sample is fragmented, electrophoretically separated on a matrix gel, and transferred to a membrane filter. The filter bound DNA or RNA is subject to hybridization with a labelled probe complementary to the sequence of interest. Hybridized probe bound to the filter is detected. A variant of the procedure is the reverse Northern blot, in which the substrate nucleic acid that is affixed to the membrane is a collection of isolated DNA fragments and the probe is RNA extracted from a tissue and labelled.

The expression level of a gene as used herein refers to the absolute or relative amount of gene product preferably transcriptional product (RNA) in a given sample. Expressed genes include genes that are transcribed into mRNA and then translated into protein, as well as genes that are transcribed into mRNA, or other types of RNA such as, tRNA, rRNA or other non-coding RNAs, that are not translated into protein. RNA expression is a highly specific process which can be monitored by detecting the absolute or relative RNA levels. Thus, the expression level refers to the amount of RNA in a sample. The expression level is usually detected using microarrays, Northern blotting, RT-PCR, SAGE, RNA-seq, or similar RNA detection methods.

When expression levels of a specific RNA in a test sample is compared to a reference sample they can either be different or equal. However, using today's detection techniques is an exact definition of different or equal result can be difficult because of noise and variations in obtained expression levels from different samples. Hence, the usual method for evaluating whether two or more expression levels are different or equal involves statistics. Statistics enables evaluation of significantly different expression levels and significantly equal expressions levels. Statistical methods involve applying a function/statistical algorithm to a set of data. Statistical theory defines a statistic as a function of a sample where the function itself is independent of the sample's distribution: the term is used both for the function and for the value of the function on a given sample. Commonly used statistical tests or methods applied to a data set include t-test, f-test or even more advanced test and methods of comparing data. Using such a test or methods enables a conclusion of whether two or more samples are significantly different or significantly equal.

In some embodiments, dosage of chromosomal regions and/or genes of the present invention, as well expression of the genes, is detected by an amplification method. Chromosomal regions, genes, and mRNA for expressed genes may be amplified prior to or simultaneous with detection. Illustrative non-limiting examples of nucleic acid amplification techniques include, but are not limited to, polymerase chain reaction (PCR), reverse transcription polymerase chain reaction (RT-PCR), transcription-mediated amplification (TMA), ligase chain reaction (LCR), strand displacement amplification (SDA), and nucleic acid sequence based amplification (NASBA). Those of ordinary skill in the art will recognize that certain amplification techniques (e.g., PCR) require that RNA be reversed transcribed to DNA prior to amplification (e.g., RT-PCR), whereas other amplification techniques directly amplify RNA (e.g., TMA and NASBA).

The polymerase chain reaction (U.S. Pat. Nos. 4,683,195, 4,683,202, 4,800,159 and 4,965,188, each of which is herein incorporated by reference in its entirety), commonly referred to as PCR, uses multiple cycles of denaturation, annealing of primer pairs to opposite strands, and primer extension to exponentially increase copy numbers of a target nucleic acid sequence. In a variation called RT-PCR, reverse transcriptase (RT) is used to make a complementary DNA (cDNA) from mRNA, and the cDNA is then amplified by PCR to produce multiple copies of DNA. For other various permutations of PCR see, e.g., U.S. Pat. Nos. 4,683,195, 4,683,202 and 4,800,159; Mullis et al., Meth. Enzymol. 155: 335 (1987); and, Murakawa et al., DNA 7: 287 (1988), each of which is herein incorporated by reference in its entirety.

Transcription mediated amplification (U.S. Pat. Nos. 5,480,784 and 5,399,491, each of which is herein incorporated by reference in its entirety), commonly referred to as TMA, synthesizes multiple copies of a target nucleic acid sequence autocatalytically under conditions of substantially constant temperature, ionic strength, and pH in which multiple RNA copies of the target sequence autocatalytically generate additional copies. See, e.g., U.S. Pat. Nos. 5,399,491 and 5,824,518, each of which is herein incorporated by reference in its entirety. In a variation described in U.S. Publ. No. 20060046265 (herein incorporated by reference in its entirety), TMA optionally incorporates the use of blocking moieties, terminating moieties, and other modifying moieties to improve TMA process sensitivity and accuracy.

The ligase chain reaction (Weiss, R., Science 254: 1292 (1991), herein incorporated by reference in its entirety), commonly referred to as LCR, uses two sets of complementary DNA oligonucleotides that hybridize to adjacent regions of the target nucleic acid. The DNA oligonucleotides are covalently linked by a DNA ligase in repeated cycles of thermal denaturation, hybridization and ligation to produce a detectable double-stranded ligated oligonucleotide product.

Strand displacement amplification (Walker, G. et al., Proc. Natl. Acad. Sci. USA 89: 392-396 (1992); U.S. Pat. Nos. 5,270,184 and 5,455,166, each of which is herein incorporated by reference in its entirety), commonly referred to as SDA, uses cycles of annealing pairs of primer sequences to opposite strands of a target sequence, primer extension in the presence of a dNTPs to produce a duplex hemiphosphorothioated primer extension product, endonuclease-mediated nicking of a hemimodified restriction endonuclease recognition site, and polymerase-mediated primer extension from the 3′ end of the nick to displace an existing strand and produce a strand for the next round of primer annealing, nicking and strand displacement, resulting in geometric amplification of product. Thermophilic SDA (tSDA) uses thermophilic endonucleases and polymerases at higher temperatures in essentially the same method (EP Pat. No. 0 684 315).

Other amplification methods include, for example: nucleic acid sequence based amplification (U.S. Pat. No. 5,130,238, herein incorporated by reference in its entirety), commonly referred to as NASBA; one that uses an RNA replicase to amplify the probe molecule itself (Lizardi et al., BioTechnol. 6: 1197 (1988), herein incorporated by reference in its entirety), commonly referred to as Q-beta replicase; a transcription based amplification method (Kwoh et al., Proc. Natl. Acad. Sci. USA 86:1173 (1989)); and, self-sustained sequence replication (Guatelli et al., Proc. Natl. Acad. Sci. USA 87: 1874 (1990), each of which is herein incorporated by reference in its entirety). For further discussion of known amplification methods see Persing, David H., “In Vitro Nucleic Acid Amplification Techniques” in Diagnostic Medical Microbiology: Principles and Applications (Persing et al., Eds.), pp. 51-87 (American Society for Microbiology, Washington, D.C. (1993)).

Non-amplified or amplified chromosomal regions, genes and mRNA can be detected by any conventional means. For example, cancer markers can be detected by hybridization with a detectably labelled probe and measurement of the resulting hybrids. Illustrative non-limiting examples of detection methods are described below.

One illustrative detection method, the Hybridization Protection Assay (HPA) involves hybridizing a chemiluminescent oligonucleotide probe (e.g., an acridinium ester-labeled (AE) probe) to the target sequence, selectively hydrolyzing the chemiluminescent label present on unhybridized probe, and measuring the chemiluminescence produced from the remaining probe in a luminometer. See, e.g., U.S. Pat. No. 5,283,174 and Norman C. Nelson et al., Nonisotopic Probing, Blotting, and Sequencing, ch. 17 (Larry J. Kricka ed., 2d ed. 1995, each of which is herein incorporated by reference in its entirety).

Another illustrative detection method provides for quantitative evaluation of the amplification process in real-time. Evaluation of an amplification process in “real-time” involves determining the amount of amplicon in the reaction mixture either continuously or periodically during the amplification reaction, and using the determined values to calculate the amount of target sequence initially present in the sample. A variety of methods for determining the amount of initial target sequence present in a sample based on real-time amplification are well known in the art. These include methods disclosed in U.S. Pat. Nos. 6,303,305 and 6,541,205, each of which is herein incorporated by reference in its entirety. Another method for determining the quantity of target sequence initially present in a sample, but which is not based on a real-time amplification, is disclosed in U.S. Pat. No. 5,710,029, herein incorporated by reference in its entirety.

Amplification products may be detected in real-time through the use of various self-hybridizing probes, most of which have a stem-loop structure. Such self-hybridizing probes are labeled so that they emit differently detectable signals, depending on whether the probes are in a self-hybridized state or an altered state through hybridization to a target sequence. By way of non-limiting example, “molecular torches” are a type of self-hybridizing probe that includes distinct regions of self-complementarity (referred to as “the target binding domain” and “the target closing domain”) which are connected by a joining region (e.g., non-nucleotide linker) and which hybridize to each other under predetermined hybridization assay conditions. In a preferred embodiment, molecular torches contain single-stranded base regions in the target binding domain that are from 1 to about 20 bases in length and are accessible for hybridization to a target sequence present in an amplification reaction under strand displacement conditions. Under strand displacement conditions, hybridization of the two complementary regions, which may be fully or partially complementary, of the molecular torch is favored, except in the presence of the target sequence, which will bind to the single-stranded region present in the target binding domain and displace all or a portion of the target closing domain. The target binding domain and the target closing domain of a molecular torch include a detectable label or a pair of interacting labels (e.g., luminescent/quencher) positioned so that a different signal is produced when the molecular torch is self-hybridized than when the molecular torch is hybridized to the target sequence, thereby permitting detection of probe:target duplexes in a test sample in the presence of unhybridized molecular torches. Molecular torches and a variety of types of interacting label pairs are disclosed in U.S. Pat. No. 6,534,274, herein incorporated by reference in its entirety.

In some embodiments, chromosomal regions, genes or mRNA are detected with a TaqMan assay (PE Biosystems, Foster City, Calif.; See e.g., U.S. Pat. No. 5,538,848 which is herein incorporated by reference). In some preferred embodiments, gene expression is assayed with a TaqMan assay. The assay is performed during a PCR reaction. The TaqMan assay exploits the 5′-3′ exonuclease activity of the AMPLITAQ GOLD DNA polymerase. A probe, specific for a given allele or mutation, is included in the PCR reaction. The probe consists of an oligonucleotide with a 5′-reporter dye (e.g., a fluorescent dye) and a 3′-quencher dye. During PCR, if the probe is bound to its target, the 5′-3′ nucleolytic activity of the AMPLITAQ GOLD polymerase cleaves the probe between the reporter and the quencher dye. The separation of the reporter dye from the quencher dye results in an increase of fluorescence. The signal accumulates with each cycle of PCR and can be monitored with a fluorimeter.

Oligonucleotide probes can be synthesized by a number of approaches, e.g. Ozaki et at, Nucleic Acids Research, 20:5205-5214 (1992); Agrawal et at, Nucleic Acids Research, 18:5419-5423 (1990); or the like. The oligonucleotide probes are conveniently synthesized on an automated DNA synthesizer, e.g. an Applied Biosystems, Inc. Foster City, Calif.) model 392 or 394 DNA/RNA Synthesizer, using standard chemistries, such as phosphoramidite chemistry, e.g. disclosed in the following references: Beaucage and Iyer, Tetrahedron, 48:2223-2311 (1992); Molko et al, U.S. Pat. Nos. 4,980,460; Koster et al, U.S. Pat. No. 4,725,677; Caruthers et al, U.S. Pat. Nos. 4,415,732; 4,458,066; and 4,973,679; and the like. Alternative chemistries, e.g. resulting in non-natural backbone groups, such as phosphorothioate, phosphoramidate, and the like, may also be employed provided that the hybridization efficiencies of the resulting oligonucleotides and/or cleavage efficiency of the exonuclease employed are not adversely affected. Preferably, the oligonucleotide probe is in the range of 15-60 nucleotides in length. More preferably, the oligonucleotide probe is in the range of 18-30 nucleotides in length. The precise sequence and length of an oligonucleotide probe depends in part on the nature of the target polynucleotide to which it binds. The binding location and length may be varied to achieve appropriate annealing and melting properties for a particular embodiment. Guidance for making such design choices can be found in many of the above-cited references describing the “TaqMan” type of assays.

Preferably, the 3′ terminal nucleotide of the oligonucleotide probe is blocked or rendered incapable of extension by a nucleic acid polymerase. Such blocking is conveniently carried out by the attachment of a reporter or quencher molecule to the terminal 3′ carbon of the oligonucleotide probe by a linking moiety.

Preferably, reporter molecules are fluorescent organic dyes derivatized for attachment to the terminal 3′ carbon or terminal 5′ carbon of the probe via a linking moiety. Preferably, quencher molecules are also organic dyes, which may or may not be fluorescent, depending on the embodiment of the invention. For example, in a preferred embodiment of the invention, the quencher molecule is fluorescent. Generally, whether the quencher molecule is fluorescent or simply releases the transferred energy from the reporter by non-radiative decay, the absorption band of the quencher should substantially overlap the fluorescent emission band of the reporter molecule. Non-fluorescent quencher molecules that absorb energy from excited reporter molecules, but which do not release the energy radiatively, are referred to herein as chromogenic molecules.

There is a great deal of practical guidance available in the literature for selecting appropriate reporter-quencher pairs for particular probes, as exemplified by the following references: Clegg (cited above); Wu et al (cited above); Pesce et at, editors, Fluorescence Spectroscopy (Marcel Dekker, New York, 1971); White et at, Fluorescence Analysis: A Practical Approach (Marcel Dekker, New York, 1970); and the like. The literature also includes references providing exhaustive lists of fluorescent and chromogenic molecules and their relevant optical properties for choosing reporter-quencher pairs, e.g. Berlman, Handbook of Fluorescence Sprectra of Aromatic Molecules, 2nd Edition (Academic Press, New York, 1971); Griffiths, Colour and Consitution of Organic Molecules (Academic Press, New York, 1976); Bishop, editor, Indicators (Pergamon Press, Oxford, 1972); Haugland, Handbook of Fluorescent Probes and Research Chemicals (Molecular Probes, Eugene, 1992); Pringsheim, Fluorescence and Phosphorescence (Interscience Publishers, New York, 1949); and the like. Further, there is extensive guidance in the literature for derivatizing reporter and quencher molecules for covalent attachment via common reactive groups that can be added to an oligonucleotide, as exemplified by the following references: Haugland (cited above); Ullman et al, U.S. Pat. No. 3,996,345; Khanna et al, U.S. Pat. No. 4,351,760; and the like.

Exemplary reporter-quencher pairs may be selected from xanthene dyes, including fluoresceins, and rhodamine dyes. Many suitable forms of these compounds are widely available commercially with substituents on their phenyl moieties which can be used as the site for bonding or as the bonding functionality for attachment to an oligonucleotide. Another group of fluorescent compounds are the naphthylamines, having an amino group in the alpha or beta position. Included among such naphthylamino compounds are 1-dimethylaminonaphthyl-5-sulfonate, 1-anilino-8-naphthalene sulfonate and 2-p-touidinyl-6-naphthalene sulfonate. Other dyes include 3-phenyl-7-isocyanatocoumarin, acridines, such as 9-isothiocyanatoacridine and acridine orange; N-(p-(2-benzoxazolyl)phenyl)maleimide; benzoxadiazoles, stilbenes, pyrenes, and the like.

Preferably, reporter and quencher molecules are selected from fluorescein and rhodamine dyes. These dyes and appropriate linking methodologies for attachment to oligonucleotides are described in many references, e.g. Khanna et al (cited above); Marshall, Histochemical J., 7:299-303 (1975); Mechnen et at, U.S. Pat. No. 5,188,934; Menchen et al, European pat. No. application 87310256.0; and Bergot et al, International application PCT/US90/05565. The latter four documents are hereby incorporated by reference.

In some embodiments, expression of the desired gene is assayed by detecting the protein encoded by the gene, preferably by an immunoassay. Illustrative non-limiting examples of immunoassays include, but are not limited to immunoprecipitation; Western blot; ELISA; immunohistochemistry; immunocytochemistry; flow cytometry; and, immuno-PCR. Polyclonal or monoclonal antibodies detectably labeled using various techniques known to those of ordinary skill in the art (e.g., colorimetric, fluorescent, chemiluminescent or radioactive) are suitable for use in the immunoassays.

Immunoprecipitation is the technique of precipitating an antigen out of solution using an antibody specific to that antigen. The process can be used to identify protein complexes present in cell extracts by targeting a protein believed to be in the complex. The complexes are brought out of solution by insoluble antibody-binding proteins isolated initially from bacteria, such as Protein A and Protein G. The antibodies can also be coupled to sepharose beads that can easily be isolated out of solution. After washing, the precipitate can be analyzed using mass spectrometry, Western blotting, or any number of other methods for identifying constituents in the complex.

A Western blot, or immunoblot, is a method to detect protein in a given sample of tissue homogenate or extract. It uses gel electrophoresis to separate denatured proteins by mass. The proteins are then transferred out of the gel and onto a membrane, typically polyvinyldifluoride or nitrocellulose, where they are probed using antibodies specific to the protein of interest. As a result, researchers can examine the amount of protein in a given sample and compare levels between several groups.

An ELISA, short for Enzyme-Linked ImmunoSorbent Assay, is a biochemical technique to detect the presence of an antibody or an antigen in a sample. It utilizes a minimum of two antibodies, one of which is specific to the antigen and the other of which is coupled to an enzyme. The second antibody will cause a chromogenic or fluorogenic substrate to produce a signal. Variations of ELISA include sandwich ELISA, competitive ELISA, and ELISPOT. Because the ELISA can be performed to evaluate either the presence of antigen or the presence of antibody in a sample, it is a useful tool both for determining serum antibody concentrations and also for detecting the presence of antigen.

Immunohistochemistry and immunocytochemistry refer to the process of localizing proteins in a tissue section or cell, respectively, via the principle of antigens in tissue or cells binding to their respective antibodies. Visualization is enabled by tagging the antibody with color producing or fluorescent tags. Typical examples of color tags include, but are not limited to, horseradish peroxidase and alkaline phosphatase. Typical examples of fluorophore tags include, but are not limited to, fluorescein isothiocyanate (FITC) or phycoerythrin (PE).

Flow cytometry is a technique for counting, examining and sorting microscopic particles suspended in a stream of fluid. It allows simultaneous multiparametric analysis of the physical and/or chemical characteristics of single cells flowing through an optical/electronic detection apparatus. A beam of light (e.g., a laser) of a single frequency or color is directed onto a hydrodynamically focused stream of fluid. A number of detectors are aimed at the point where the stream passes through the light beam; one in line with the light beam (Forward Scatter or FSC) and several perpendicular to it (Side Scatter (SSC) and one or more fluorescent detectors). Each suspended particle passing through the beam scatters the light in some way, and fluorescent chemicals in the particle may be excited into emitting light at a lower frequency than the light source. The combination of scattered and fluorescent light is picked up by the detectors, and by analyzing fluctuations in brightness at each detector, one for each fluorescent emission peak, it is possible to deduce various facts about the physical and chemical structure of each individual particle. FSC correlates with the cell volume and SSC correlates with the density or inner complexity of the particle (e.g., shape of the nucleus, the amount and type of cytoplasmic granules or the membrane roughness).

Immuno-polymerase chain reaction (IPCR) utilizes nucleic acid amplification techniques to increase signal generation in antibody-based immunoassays. Because no protein equivalence of PCR exists, that is, proteins cannot be replicated in the same manner that nucleic acid is replicated during PCR, the only way to increase detection sensitivity is by signal amplification. The target proteins are bound to antibodies which are directly or indirectly conjugated to oligonucleotides. Unbound antibodies are washed away and the remaining bound antibodies have their oligonucleotides amplified. Protein detection occurs via detection of amplified oligonucleotides using standard nucleic acid detection methods, including real-time methods.

In some embodiments, a computer-based analysis program is used to translate the raw data generated by the detection assay (e.g., the presence, absence, or amount of a given marker or markers) into data of predictive value for a clinician. The clinician can access the predictive data using any suitable means. Thus, in some preferred embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, information provides, medical personal, and subjects. For example, in some embodiments of the present invention, a sample (e.g., a biopsy sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (i.e., expression data), specific for the diagnostic or prognostic information desired for the subject.

The profile data is then prepared in a format suitable for interpretation by a treating clinician. For example, rather than providing raw expression data, the prepared format may represent a diagnosis or risk assessment (e.g., likelihood of cancer being present) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may chose further intervention or counselling based on the results. In some embodiments, the data is used for research use. For example, the data may be used to further optimize the inclusion or elimination of markers as useful indicators of a particular condition or stage of disease.

Kits

Compositions for use in the diagnostic methods of embodiments of the present invention include, but are not limited to, probes, amplification oligonucleotides, and antibodies. Any of these compositions, alone or in combination with other compositions, may be provided in the form of a kit. For example, the single labeled probe and pair of amplification oligonucleotides may be provided in a kit for the amplification and detection of cancer markers. Kits may further comprise appropriate controls and/or detection reagents. The probe and antibody compositions may also be provided in the form of an array.

In still other embodiments, the kits comprise at least one vial containing a control analyte or analytes (such as a genomic sequence). In still other embodiments, the kit comprises instructions for using the reagents contained in the kit for the detection of at least one type of analyte. In some embodiments, the instructions further comprise the statement of intended use required by the U.S. Food and Drug Administration (FDA) in labelling in vitro diagnostic products. The FDA classifies in vitro diagnostics as medical devices and requires that they be approved through the 510(K) procedure. Information required in an application under 510(k) includes: 1) The in vitro diagnostic product name, including the trade or proprietary name, the common or usual name, and the classification name of the device; 2) The intended use of the product; 3) The establishment registration number, if applicable, of the owner or operator submitting the 510(k) submission; the class in which the in vitro diagnostic product is placed under section 513 of the FD&C Act, if known, its appropriate panel, or, if the owner or operator determines that the device has not been classified under such section, a statement of that determination and the basis for the determination that the in vitro diagnostic product is not so classified; 4) Proposed labels, labelling and advertisements sufficient to describe the in vitro diagnostic product, its intended use, and directions for use. Where applicable, photographs or engineering drawings should be supplied; 5) A statement indicating that the device is similar to and/or different from other in vitro diagnostic products of comparable type in commercial distribution in the U.S., accompanied by data to support the statement; 6) A 510(k) summary of the safety and effectiveness data upon which the substantial equivalence determination is based; or a statement that the 510(k) safety and effectiveness information supporting the FDA finding of substantial equivalence will be made available to any person within 30 days of a written request; 7) A statement that the submitter believes, to the best of their knowledge, that all data and information submitted in the premarket notification are truthful and accurate and that no material fact has been omitted; 8) Any additional information regarding the in vitro diagnostic product requested that is necessary for the FDA to make a substantial equivalency determination.

Drug Screening

In some embodiments, the present invention provides drug screening assays (e.g., to screen for anticancer drugs). The screening methods utilize cancer markers identified using the methods described herein. For example, in some embodiments, the present invention provides methods of screening for compounds that modulate (e.g., increase or decrease) the expression of cancer marker genes. The compounds or agents may modulate transcription, by interacting, for example, with the promoter region. The compounds or agents may modulate mRNA produced from the cancer markers (e.g., by RNA interference, antisense technologies, etc.). The compounds or agents may modulate pathways that are upstream or downstream of the biological activity of the cancer marker. In some embodiments, candidate compounds are antisense or interfering RNA agents (e.g., oligonucleotides) directed against cancer markers. In other embodiments, candidate compounds are antibodies or small molecules that specifically bind to a cancer marker regulator or expression products to modulate biological function.

In one screening method, candidate compounds are evaluated for their ability to modulate cancer marker expression by contacting a compound with a cell expressing a cancer marker and then assaying for the effect of the candidate compounds on expression. In some embodiments, the effect of candidate compounds on expression of a cancer marker gene is assayed for by detecting the level of cancer marker mRNA expressed by the cell. mRNA expression can be detected by any suitable method. In other embodiments, the effect of candidate compounds on expression of cancer marker genes is assayed by measuring the level of polypeptide encoded by the cancer markers. The level of polypeptide expressed can be measured using any suitable method, including but not limited to, those disclosed herein.

Specifically, the present invention provides screening methods for identifying modulators, e.g., candidate or test compounds or agents (e.g., proteins, peptides, peptidomimetics, peptoids, small molecules or other drugs) which bind to cancer markers of embodiments of the present invention, have an inhibitory (or stimulatory) effect on, for example, cancer marker expression or cancer marker activity, or have a stimulatory or inhibitory effect on, for example, the expression or activity of a cancer marker substrate. Compounds thus identified can be used to modulate the activity of target gene products (e.g., cancer marker genes) either directly or indirectly in a therapeutic protocol, to elaborate the biological function of the target gene product, or to identify compounds that disrupt normal target gene interactions. Compounds that inhibit the activity or expression of cancer markers are useful in the treatment of proliferative disorders, e.g., cancer, particularly prostate cancer.

In one embodiment, the invention provides assays for screening candidate or test compounds that are substrates of a cancer marker protein or polypeptide or a biologically active portion thereof. In another embodiment, the invention provides assays for screening candidate or test compounds that bind to or modulate the activity of a cancer marker protein or polypeptide or a biologically active portion thereof.

The test compounds can be obtained using any of the numerous approaches in combinatorial library methods known in the art, including biological libraries; peptoid libraries (libraries of molecules having the functionalities of peptides, but with a novel, non-peptide backbone, which are resistant to enzymatic degradation but which nevertheless remain bioactive; see, e.g., Zuckennann et al., J. Med. Chem. 37: 2678-85 [1994]); spatially addressable parallel solid phase or solution phase libraries; synthetic library methods requiring deconvolution; the ‘one-bead one-compound’ library method; and synthetic library methods using affinity chromatography selection. The biological library and peptoid library approaches are preferred for use with peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al., Proc. Natl. Acad. Sci. U.S.A. 90:6909 [1993]; Erb et al., Proc. Nad. Acad. Sci. USA 91:11422 [1994]; Zuckermann et al., J. Med. Chem. 37:2678 [1994]; Cho et al., Science 261:1303 [1993]; Carrell et al., Angew. Chem. Int. Ed. Engl. 33.2059 [1994]; Carell et al., Angew. Chem. Int. Ed. Engl. 33:2061 [1994]; and Gallop et al., J. Med. Chem. 37:1233 [1994].

Libraries of compounds may be presented in solution (e.g., Houghten, Biotechniques 13:412-421 [1992]), or on beads (Lam, Nature 354:82-84 [1991]), chips (Fodor, Nature 364:555-556 [1993]), bacteria or spores (U.S. Pat. No. 5,223,409; herein incorporated by reference), plasmids (Cull et al., Proc. Nad. Acad. Sci. USA 89:18651869 [1992]) or on phage (Scott and Smith, Science 249:386-390 [1990]; Devlin Science 249:404-406 [1990]; Cwirla et al., Proc. Natl. Acad. Sci. 87:6378-6382 [1990]; Felici, J. Mol. Biol. 222:301 [1991]).

In one embodiment, an assay is a cell-based assay in which a cell that expresses a cancer marker mRNA or protein or biologically active portion thereof is contacted with a test compound, and the ability of the test compound to the modulate cancer marker's activity is determined Determining the ability of the test compound to modulate cancer marker activity can be accomplished by monitoring, for example, changes in enzymatic activity, destruction or mRNA, or the like.

The ability of the test compound to modulate cancer marker binding to a compound, e.g., a cancer marker substrate or modulator, can also be evaluated. This can be accomplished, for example, by coupling the compound, e.g., the substrate, with a radioisotope or enzymatic label such that binding of the compound, e.g., the substrate, to a cancer marker can be determined by detecting the labeled compound, e.g., substrate, in a complex.

Alternatively, the cancer marker is coupled with a radioisotope or enzymatic label to monitor the ability of a test compound to modulate cancer marker binding to a cancer marker substrate in a complex. For example, compounds (e.g., substrates) can be labeled with ¹²⁵I, ³⁵S ¹⁴C or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

The ability of a compound (e.g., a cancer marker substrate) to interact with a cancer marker with or without the labelling of any of the interactants can be evaluated. For example, a microphysiometer can be used to detect the interaction of a compound with a cancer marker without the labelling of either the compound or the cancer marker (McConnell et al. Science 257:1906-1912 [1992]). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and cancer markers.

In yet another embodiment, a cell-free assay is provided in which a cancer marker protein or biologically active portion thereof is contacted with a test compound and the ability of the test compound to bind to the cancer marker protein, mRNA, or biologically active portion thereof is evaluated. Preferred biologically active portions of the cancer marker proteins or mRNA to be used in assays include fragments that participate in interactions with substrates or other proteins, e.g., fragments with high surface probability scores.

Cell-free assays involve preparing a reaction mixture of the target gene protein and the test compound under conditions and for a time sufficient to allow the two components to interact and bind, thus forming a complex that can be removed and/or detected.

The interaction between two molecules can also be detected, e.g., using fluorescence energy transfer (FRET) (see, for example, Lakowicz et al., U.S. Pat. No. 5,631,169; Stavrianopoulos et al., U.S. Pat. No. 4,968,103; each of which is herein incorporated by reference). A fluorophore label is selected such that a first donor molecule's emitted fluorescent energy will be absorbed by a fluorescent label on a second, ‘acceptor’ molecule, which in turn is able to fluoresce due to the absorbed energy.

Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label should be maximal. A FRET binding event can be conveniently measured through standard fluorometric detection means well known in the art (e.g., using a fluorimeter).

In another embodiment, determining the ability of the cancer marker protein or mRNA to bind to a target molecule can be accomplished using real-time Biomolecular Interaction Analysis (BIA) (see, e.g., Sjolander and Urbaniczky, Anal. Chem. 63:2338-2345 [1991] and Szabo et al. Curr. Opin. Struct. Biol. 5:699-705 [1995]). “Surface plasmon resonance” or “BIA” detects biospecific interactions in real time, without labelling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal that can be used as an indication of real-time reactions between biological molecules.

In one embodiment, the target gene product or the test substance is anchored onto a solid phase. The target gene product/test compound complexes anchored on the solid phase can be detected at the end of the reaction. Preferably, the target gene product can be anchored onto a solid surface, and the test compound, (which is not anchored), can be labeled, either directly or indirectly, with detectable labels discussed herein.

It may be desirable to immobilize cancer markers, an anti-cancer marker antibody or its target molecule to facilitate separation of complexed from non-complexed forms of one or both of the proteins, as well as to accommodate automation of the assay. Binding of a test compound to a cancer marker protein, or interaction of a cancer marker protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase-cancer marker fusion proteins or glutathione-S-transferase/target fusion proteins can be adsorbed onto glutathione Sepharose beads (Sigma Chemical, St. Louis, Mo.) or glutathione-derivatized microtiter plates, which are then combined with the test compound or the test compound and either the non-adsorbed target protein or cancer marker protein, and the mixture incubated under conditions conducive for complex formation (e.g., at physiological conditions for salt and pH). Following incubation, the beads or microtiter plate wells are washed to remove any unbound components, the matrix immobilized in the case of beads, complex determined either directly or indirectly, for example, as described above.

Alternatively, the complexes can be dissociated from the matrix, and the level of cancer markers binding or activity determined using standard techniques. Other techniques for immobilizing either cancer markers protein or a target molecule on matrices include using conjugation of biotin and streptavidin. Biotinylated cancer marker protein or target molecules can be prepared from biotin-NHS(N-hydroxy-succinimide) using techniques known in the art (e.g., biotinylation kit, Pierce Chemicals, Rockford, EL), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-IgG antibody).

This assay is performed utilizing antibodies reactive with cancer marker protein or target molecules but which do not interfere with binding of the cancer markers protein to its target molecule. Such antibodies can be derivatized to the wells of the plate, and unbound target or cancer markers protein trapped in the wells by antibody conjugation. Methods for detecting such complexes, in addition to those described above for the GST-immobilized complexes, include immunodetection of complexes using antibodies reactive with the cancer marker protein or target molecule, as well as enzyme-linked assays which rely on detecting an enzymatic activity associated with the cancer marker protein or target molecule.

Alternatively, cell free assays can be conducted in a liquid phase. In such an assay, the reaction products are separated from unreacted components, by any of a number of standard techniques, including, but not limited to: differential centrifugation (see, for example, Rivas and Minton, Trends Biochem Sci 18:284-7 [1993]); chromatography (gel filtration chromatography, ion-exchange chromatography); electrophoresis (see, e.g., Ausubel et al., eds. Current Protocols in Molecular Biology 1999, J. Wiley: New York.); and immunoprecipitation (see, for example, Ausubel et al., eds. Current Protocols in Molecular Biology 1999, J. Wiley: New York). Such resins and chromatographic techniques are known to one skilled in the art (See e.g., Heegaard J. Mol. Recognit. 11:141-8 [1998]; Hageand Tweed J. Chromatogr. Biomed. Sci. Appl 699:499-525 [1997]). Further, fluorescence energy transfer may also be conveniently utilized, as described herein, to detect binding without further purification of the complex from solution.

The assay can include contacting the cancer markers protein, mRNA, or biologically active portion thereof with a known compound that binds the cancer marker to form an assay mixture, contacting the assay mixture with a test compound, and determining the ability of the test compound to interact with a cancer marker protein or mRNA, wherein determining the ability of the test compound to interact with a cancer marker protein or mRNA includes determining the ability of the test compound to preferentially bind to cancer markers or biologically active portion thereof, or to modulate the activity of a target molecule, as compared to the known compound.

To the extent that cancer markers can, in vivo, interact with one or more cellular or extracellular macromolecules, such as proteins, inhibitors of such an interaction are useful. A homogeneous assay can be used can be used to identify inhibitors.

For example, a preformed complex of the target gene product and the interactive cellular or extracellular binding partner product is prepared such that either the target gene products or their binding partners are labeled, but the signal generated by the label is quenched due to complex formation (see, e.g., U.S. Pat. No. 4,109,496, herein incorporated by reference, that utilizes this approach for immunoassays). The addition of a test substance that competes with and displaces one of the species from the preformed complex will result in the generation of a signal above background. In this way, test substances that disrupt target gene product-binding partner interaction can be identified. Alternatively, cancer markers protein can be used as a “bait protein” in a two-hybrid assay or three-hybrid assay (see, e.g., U.S. Pat. No. 5,283,317; Zervos et al., Cell 72:223-232 [1993]; Madura et al., J. Biol. Chem. 268.12046-12054 [1993]; Bartel et al., Biotechniques 14:920-924 [1993]; Iwabuchi et al., Oncogene 8:1693-1696 [1993]; and Brent WO 94/10300; each of which is herein incorporated by reference), to identify other proteins, that bind to or interact with cancer markers (“cancer marker-binding proteins” or “cancer marker-bp”) and are involved in cancer marker activity. Such cancer marker-bps can be activators or inhibitors of signals by the cancer marker proteins or targets as, for example, downstream elements of a cancer markers-mediated signalling pathway.

Modulators of cancer markers expression can also be identified. For example, a cell or cell free mixture is contacted with a candidate compound and the expression of cancer marker mRNA or protein evaluated relative to the level of expression of cancer marker mRNA or protein in the absence of the candidate compound. When expression of cancer marker mRNA or protein is greater in the presence of the candidate compound than in its absence, the candidate compound is identified as a stimulator of cancer marker mRNA or protein expression. Alternatively, when expression of cancer marker mRNA or protein is less (i.e., statistically significantly less) in the presence of the candidate compound than in its absence, the candidate compound is identified as an inhibitor of cancer marker mRNA or protein expression. The level of cancer markers mRNA or protein expression can be determined by methods described herein for detecting cancer markers mRNA or protein.

A modulating agent can be identified using a cell-based or a cell free assay, and the ability of the agent to modulate the activity of a cancer markers protein can be confirmed in vivo, e.g., in an animal such as an animal model for a disease (e.g., an animal with prostate cancer or metastatic prostate cancer; or an animal harboring a xenograft of a prostate cancer from an animal (e.g., human) or cells from a cancer resulting from metastasis of a prostate cancer (e.g., to a lymph node, bone, or liver), or cells from a prostate cancer cell line.

This invention further pertains to novel agents identified by the above-described screening assays (See e.g., below description of cancer therapies). Accordingly, it is within the scope of this invention to further use an agent identified as described herein (e.g., a cancer marker modulating agent, an antisense cancer marker nucleic acid molecule, a siRNA molecule, a cancer marker specific antibody, or a cancer marker-binding partner) in an appropriate animal model (such as those described herein) to determine the efficacy, toxicity, side effects, or mechanism of action, of treatment with such an agent. Furthermore, novel agents identified by the above-described screening assays can be, e.g., used for treatments as described herein.

### EXAMPLES

### Example 1

### Identification of Eight Target Genes of the Recurrent 3p12-p14 Loss in Cervical Cancer by Integrative Fenomic Profiling

### Materials and Methods

Patients and Samples

Samples from 4 CIN2, 39 CIN3, and 6 CIN3 adjacent to invasive carcinoma (SCCCIN3) collected at VU University Medical Center in The Netherlands, and 166 invasive squamous cell carcinoma of the uterine cervix (Table 4), collected at the Norwegian Radium Hospital in Norway, were included in the study. The clinical protocols were approved by the Institutional Review Board of the VU University Medical Center and the Regional Committee for Medical Research Ethics in southern Norway, respectively. Written informed consent was obtained from all patients.

Array Comparative Genomic Hybridization

All 49 CIN lesions were subjected to aCGH analysis. Microdissected DNA was amplified and hybridized to 105K arrays (Agilent Technologies, Palo Alto, USA) using a pool of five normal cervical tissues as reference. Array data are available from the Gene Expression Omnibus (GEO) through the accession numbers GSE31241 (CIN2/3) and GSE30155 (SCC-CIN3). A total of 92 invasive carcinomas were included in aCGH experiments (Table 4). DNA was cohybridized with normal female DNA to array slides containing 4559 unique genomic BAC and PAC clones. Array data are available from the ArrayExpress repository through the accession number E-MTAB-1161.

### Gene Expression Arrays

Illumina gene expression profiling of 77 invasive carcinomas with aCGH data (integrative cohort; Table 4), 74 invasive carcinomas without aCGH data (validation cohort), and the cervical cancer cell lines HeLa, SiHa, and CaSki was performed using the Illumina beadarrays HumanWG-6 v3 (tumors, HeLa) and HumanHT-12 v4 (SiHa, CaSki), with 48 000 transcripts (Illumina Inc., San Diego, Calif.). The Illumina data are available in GEO (GSE38964). cDNA microarrays were used on 90 of the invasive carcinoma samples with aCGH data to confirm the Illumina data of selected genes (ArrayExpress accession no. E-MTAB-1199).

Immunohistochemistry

Formalin-fixed, paraffin-embedded tissue sections from 86 invasive carcinomas with aCGH data and the cervical cancer cell lines HeLa, SiHa, and CaSki were immunostained with RYBP rabbit polyclonal antibody (LS-C80245, 1:4000) from LifeSpan BioSciences (Seattle, Wash.) and TMF1 rabbit polyclonal antibody (HPA008729, 1:700) from Sigma-Aldrich (Missouri, Mo.), using the Dako EnVision™ FLEX+ detection system (Dako Corp., Glostrup, Denmark) manually.

Cell Culture, siRNA Transfection, and Western Blotting

Knockdown of the candidate target genes RYBP, TMF1, and PSMD6 was performed in the HeLa, SiHa, and CaSki cervical cancer cell lines, which harbor no chromosomal loss on 3p (Lockwood et al., Int J Cancer 2007; 120:436-443). For Western blotting, RYBP rabbit polyclonal antibody (LS-C80245, 1:4000), TMF 1 rabbit polyclonal antibody (HPA008729, 1:700), and goat anti-rabbit IgGs (Dako Corp., Glostrup, Denmark) were used. A PSMD6 antibody showing satisfactorily specificity was not available.

Statistics

Univariate Cox regression analysis was used to identify the 3p region that was associated with clinical outcome of the patients with invasive carcinoma. Survival curves were generated by Kaplan-Meier analysis and compared using log-rank test. Pvalues<0.05 were considered significant. Spearman's rank correlation analysis was used to search for significant correlations between gene dosage and expression. The analysis was based on semi-discrete data, for which gene dosages higher than 0.9 were set to 1 (Lando et al., supra). Thep-values were adjusted by the multiple testing procedure developed by Benjamini and Hochberg to control the false discovery rate (FDR) (Benjamini and Hochberg, Journal of R Stat Soc B 1995; 57:289-300), and a cut off of adjusted (adj) p<0.01 was used for selection of candidate target genes. Pathway signaling of the candidate 3p target genes was investigated by combining global network and GO analysis. Networks were constructed by selecting the known interaction partners of the proteins encoded by the candidate targets from an integrated set of 10 protein interaction databases (Razick et al., BMC Bioinformatics 2008; 9:405), whereby each interaction had at least one Medline citation, was experimentally validated, and had a physical binding interaction. The partners encoded by genes that were differentially expressed between patients with and without 3p loss, as determined with the linear models for microarray data (LIMMA) software (Smyth, Stat Appl Genet Mol Biol 2004; 3), were used as nodes in the networks. The most significant aCGH probe in the Cox regression analysis (70.7 Mb) was used to divide patients into the two groups in the LIMMA analysis. To achieve an appropriate number of genes for the analysis, a p-value of 0.1 was used as cut off value in the LIMMA analysis, leading to 5271 differentially expressed genes. A gene set analysis using the Significance Analysis of Microarrays for Gene Sets (SAM-GS) tool, which is based on the moderated t-statistic in SAM (Dinu et al., BMC Bioinformatics 2007; 8:242; Liu et al., BMC Bioinformatics 2007; 8:431) with the multiple testing algorithm described above (Benjamini et al., supra) to control FDR, was thereafter applied with 10 000 permutations to confirm a coordinate change in expression for the set of network genes. This procedure was used since pathway regulation may induce significant changes in the expression of some members, while others show only a modest change (Mootha et al., Nat Genet 2003; 34:267-273). The networks were visualized using the Cytoscape Software (Shannon et al., Genome Res 2003; 13:2498-2504). The GO analysis was performed to find biological processes that were overrepresented among the genes in the network. The GO categories of the genes were compared with those of all genes on the array using the master-target procedure with the Fisher's exact test in the eGOn software (Beisvag et al., BMC Bioinformatics 2006; 7:470), where the multiple testing procedure described above was used to control the FDR.

Patients

CIN2/3 lesions included 43 formalin-fixed paraffin-embedded specimens (4 CIN2, 39 CIN3), collected from women participating in the population based screening study POBASCAM (trial #ISRCTN20781131) and six CIN3 adjacent to invasive carcinoma (SCC-CIN), collected at the Departments of Obstetrics and Gynecology (VU University Medical Center, Amsterdam, The Netherlands) during routine clinical practice. The CIN2/3 samples were all HPV- and CDKN2A-positive, ensuring the inclusion of only CIN2/3 lesions harboring transforming infections.

Tumor specimens were achieved from 166 patients with squamous cell carcinoma (SCC) of the cervix who were prospectively recruited to the chemoradiotherapy protocol (Table 4). All patients were treated with external irradiation and brachytherapy combined with adjuvant cisplatin, and followed up as described previously. Briefly, external radiation included 50 Gy to tumor, parametria, and adjacent pelvic wall and 45 Gy to the remaining part of the pelvic region. Intracavitary brachytherapy was given as 21 Gy in five fractions to point A. Adjuvant cisplatin (40 mg/m2) was employed weekly in maximum 6 courses during the period of external radiation. The follow up involved regular clinical examinations followed by imaging in cases of symptoms of recurrent disease. Locoregional control; i.e., complete and persistent regression of tumor within the irradiated field, and progression free survival; i.e., the time between diagnosis and the first event of locoregional and/or distant relapse, were used as end points. Ten patients died of causes not related to cancer and were therefore censored. One to four biopsies were taken at different locations of the tumor at the time of diagnosis, immediately frozen in liquid nitrogen, stored at -80° C., and used for aCGH and gene expression analyses. A separate biopsy of each tumor was fixed in 4% buffered formalin and used for immunohistochemistry.

Array CGH

CIN2/3 & SCC-CIN3 To avoid contamination of the aCGH profile with DNA from surrounding normal tissue, the specimens were laser capture microdissected using a Leica ASLMD microscope (Leica Microsystems, Newcastle Upon Tyne, UK) as described previously. Dissected material was incubated overnight at 37° C. with 1 M sodium thiocyanate, washed with PBS, and treated with 1 mg/ml proteinase K for 5 days with daily enzyme additions, followed by DNA extraction using the Qiagen DNA micro kit (Qiagen, Westburg, Leusden, The Netherlands) according to the manufacturer's protocol. Whole genome amplification using the Bioscore kit (Enzo Bioscore™ Screening and Amplification, Enzo Life Sciences, Farmingdale, USA) was performed as described by Buffart et al. The amplified DNA was labeled and hybridized to 105K arrays containing 99 000 synthetic 60-mer oligonucleotides (Agilent Technologies, Palo Alto, USA), using a pool of five normal cervical tissues, without malignant cervical disease and negative for HPV, as reference. After array scanning, image analysis, spot filtering, and ratio normalization, an automated algorithm for determination of gains and losses, CGHcall version 2.5.0, was used.

CERVICAL TUMORS DNA was isolated from the samples according to a standard protocol with proteinase K, phenol, chloroform, and isoamylalcohol, labeled, and co-hybridized with normal female DNA to the array slides. DNA from different biopsies of the same tumor was pooled. Array slides produced at the Microarray Facility at the Norwegian Radium Hospital, containing 4549 unique genomic BAC and PAC clones that spanned the entire human genome at approximately 1 Mb resolution, were used. After array scanning, image analysis, spot filtering, and ratio normalization, the GLAD algorithm was applied for ratio smoothing and breakpoint detection (Heselmeyer et al., Genes Chromosomes Cancer 1997; 19:233-240). GeneCount was used to transfer the smoothed ratios to absolute DNA copy numbers, by correcting for tumor ploidy and proportion of normal cells within the samples. Flow cytometry was used to determine tumor ploidy, and tumor cell fraction was estimated by GeneCount prior to the copy number calculations. The copy numbers were transferred into absolute gene dosages by dividing the data with the ploidy. Gains and losses were scored by using gene dosage thresholds of 1.1 and 0.9, respectively, taking into account an uncertainty in the ploidy measurement of approximately 10%. Losses were divided into moderate loss (ML) and severe loss (SL), where a gene dosage of less than or equal to 0.5 was required for scoring severe loss, implying that half or more of the specific genomic region was lost.

Gene Expression Arrays

RNA was isolated from the tumor samples by use of Trizol reagent (Invitrogen, Carlsbad, Calif.) and from the cell cultures by the use of RNeasy mini kit (Qiagen, Germantown, Md.). RNA from different biopsies of the same tumor was pooled.

ILLUMINA GENE EXPRESSION BEADARRA YS Amplification of RNA was performed using the Illumina® TotalPrep RNA amplification kit (Ambion Inc., Austin, Tex.), with 500 ng of total RNA as input material, and cRNA was synthesized, labeled, and hybridized to the arrays. The hybridized arrays were stained with streptavidin-Cy3 (Amersham™, PA43001, Buckinghampshire, UK) and scanned with an Illumina Beadarray reader. Extraction, quality control, and quantile normalization were performed using software provided by the producer (Illumina Inc.).

cDNA MICROARRAYS The microarray slides were produced at the Microarray Facility at the Norwegian Radium Hospital and contained more than 12 000 unique cDNA clones. Total RNA was isolated from the biopsies, labeled, and co-hybridized with reference RNA (Universal Human Reference RNA, Stratagene, La Jolla, Calif.) to the array slides. All hybridizations were performed twice in a dye-swap design. After array scanning, image analysis, spot filtering, and ratio normalization, the average expression ratios were calculated from the two data sets and used in the further analyses.

Methylation-Specific PCR (MSP) Analysis

MSP analysis of RYBP, TMF1, and PSMD6 was performed on 70 invasive carcinomas in the integrative cohort. Specific primers designed to amplify the methylated DNA sequence of the promoter regions are shown in Table 5. The modified, unmethylated sequence of the housekeeping gene β-actin (ACTB) was amplified as a reference to verify sufficient DNA quality and successful DNA modification.

Immunohistochemistry

For immunostaining, the Dako EnVision™ FLEX+ detection system (Dako Corp., Glostrup, Denmark) was used manually. Heat induced epitope retrieval was performed with a PT Link using Envision™ FLEX Target Retrieval Solution at high pH (Tris/EDTA buffer pH 9). Incubation time for the primary antibodies was 45 minutes at room temperature. EnVisionTM FLEX+ Rabbit LINKER was used for signal amplification of the primary antibody, and the reaction was visualized by EnVision™ FLEX DAB+ Chromogen. Placenta and small intestine were used as positive controls for RYBP and TMF1, respectively. As negative control, the primary antibodies were substituted with normal rabbit IgG of the same concentration as the primary antibodies. The antibody staining was evaluated by an experienced scientist at the Department of Pathology (R.H.) who was blinded for the clinical data. Both the staining intensity and percentage of positive tumor cells were given a score ranging from 0-3. For the percentage of positive tumor cells, the scores were as follows: 0, 0%; 1, 1-10%; 2, 11-50%; 3, >50%. The intensity was scored as: 0, absent; 1, weak; 2, intermediate; 3, strong. The product (composite score), ranging from 0-9, was used for further analysis.

Cell Culture

The cells were grown in DMEM glutamax supplemented with 10% heat-inactivated fetal calf serum, penicillin, and streptomycin at 37° C. in a humidified atmosphere with 5% CO2. The correct identity of the cell line DNA profiles was confirmed by STR profiling using Powerplex 16 (Promega, Madison, Wis.).

siRNA Transfection

For knockdown of RYBP, TMF1, and PSMD6, the cell lines were plated 24 hours prior to transfection with siRNA. Each well received 100 nM siGENOME SMARTpool (Dharmacon, Chicago, Ill.) with four gene specific siRNAs, mixed with Oligofectamine transfection reagent (Invitrogen, Carlsbad, Calif.). Mock cells received only transfection reagent, whereas control cells were transfected with 100 nM siGENOME Non-Targeting siRNA Pool #1. The transfected cells were harvested after 72 hours.

Western Blotting

Cells were lysed in lysis buffer (20 mM TrisHCl, pH 7.5, containing 137 mM NaCl, 10% glycerol, and 1% Igepal) with protease inhibitors. Samples were separated on 8-16% Tris-HEPES-SDS polyacrylamide gels (Pierce Biotechnology, Rockford, Ill.), blotted on a PVDF membrane, and visualized using LumiGLO Chemiluminescent substrate system (KPL, Gaithersburg, Md.).

### Results

Chromosome 3p Loss in Precancerous and Cancerous Lesions

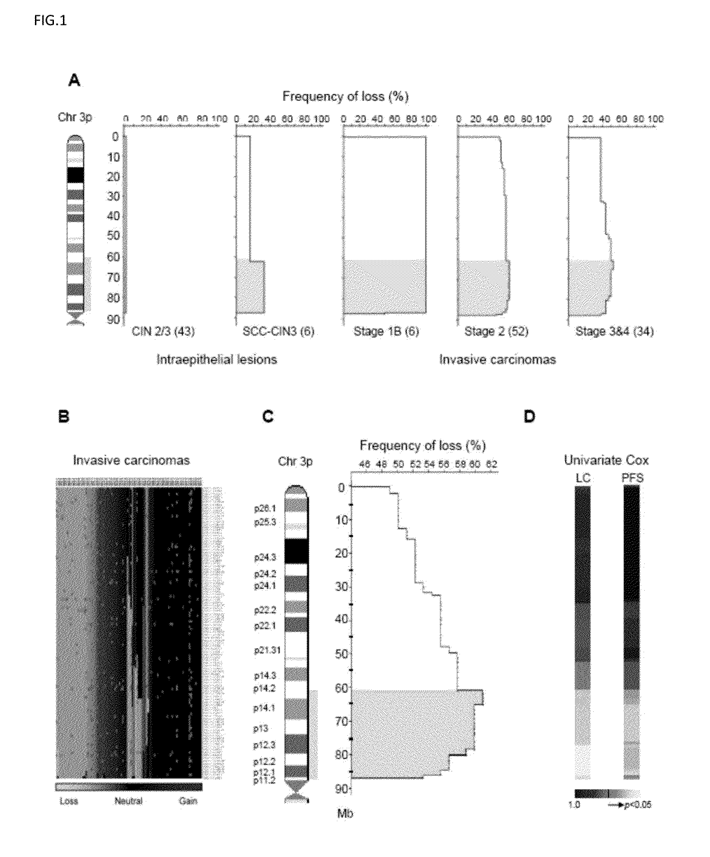

Gene copy number changes on chromosome 3p were identified from the aCGH profiles of 49 high-grade CIN lesions and 92 invasive carcinomas (integrative cohort; Table 4) and compared across the samples. A higher frequency of 3p loss was seen in the cancerous than in the precancerous lesions (FIG. 1A). Loss of the region closest to the centromere was most frequent and occurred in all stage 1b carcinomas, in 62% at stage 2, and in 53% at stages 3 and 4. In contrast, only one of 43 precancerous samples (2%; CIN3), and two of six CIN3 adjacent to invasive carcinoma (SCC-CIN3) (33%) harbored loss of this region. Hence, loss on 3p was not associated with the development of high-grade CIN, but rather with the invasive growth.

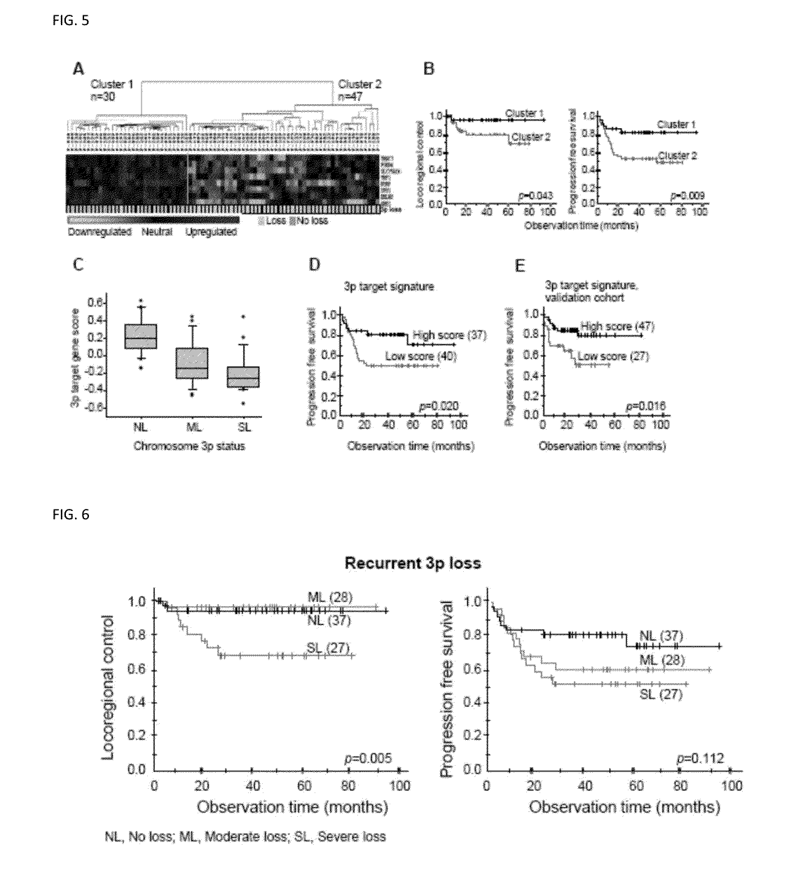

Analysis of all invasive stages combined showed 3p loss in 61% of the tumors, whereas a gain was rarely seen (13%) (FIG. 1B, C). In the majority of cases with loss (80%), the whole 3p arm was affected, but the most frequent loss extended from p12.2 to p14.2 (60.9-80.6 Mb) and involved the fragile region at p14.2. Cox regression analysis of the 3p gene dosages showed that loss of 3p11.2-p14.2, encompassing the 60.9-87.6 Mb region, was significantly associated with clinical outcome, regardless of whether locoregional control or progression free survival was used as endpoint (FIG. 1D). The locoregional relapse was more common for patients with severe loss than for those with moderate loss (FIG. 6).

Candidate 3p Target Genes

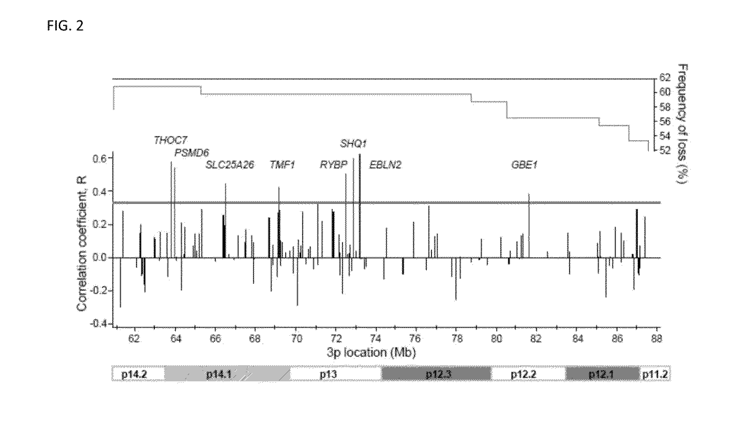

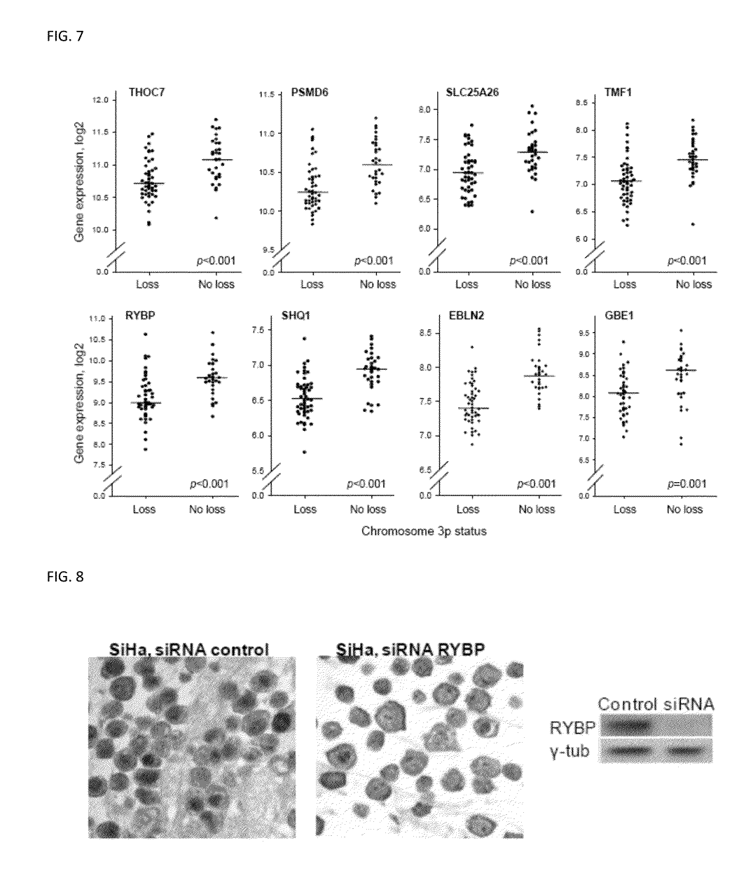

Candidate 3p target genes were searched for by performing a complete transcript mapping of 3p11.2-p14.2 in 77 of the invasive carcinomas presented in FIG. 1B-D (Table 4). The expression of eight of the 147 genes encoding proteins or hypothetical proteins within the region; i.e., THOC7, PSMD6, SLC25A26, TMF1, RYBP, SHQ1, EBLN2, and GBE1, showed a highly significant correlation to gene dosage with an adjusted p-value below the cut off level of 0.01 (p<0.001; Table 1, FIG. 2, FIG. 7). The genes were located within the 63.8-81.6 Mb region (3p12.3-p14.1), which also includes many genes with a weaker or no correlation to gene dosage, like FHIT (59.7-61.2 Mb; p=0.08), FOXP1 (71.0 Mb; p=0.05), and ROBO1 (78.6 Mb; p=0.23). Of the eight genes, GBE1 (81.5 Mb) was located outside the most frequently lost region, but was still affected in 56% of the carcinomas and included in the recurrent 60.9-81.6 Mb region that was depicted in (Lando et al., supra). The strong downregulation of the eight genes in tumors with 3p loss indicates that they were regulated primarily by the gene dosage, and they were therefore identified as candidate targets of the loss. A high degree of co-regulation was seen for all pairs of genes (p<0.05), except for RYBP versus GBE1 (p=0.3).

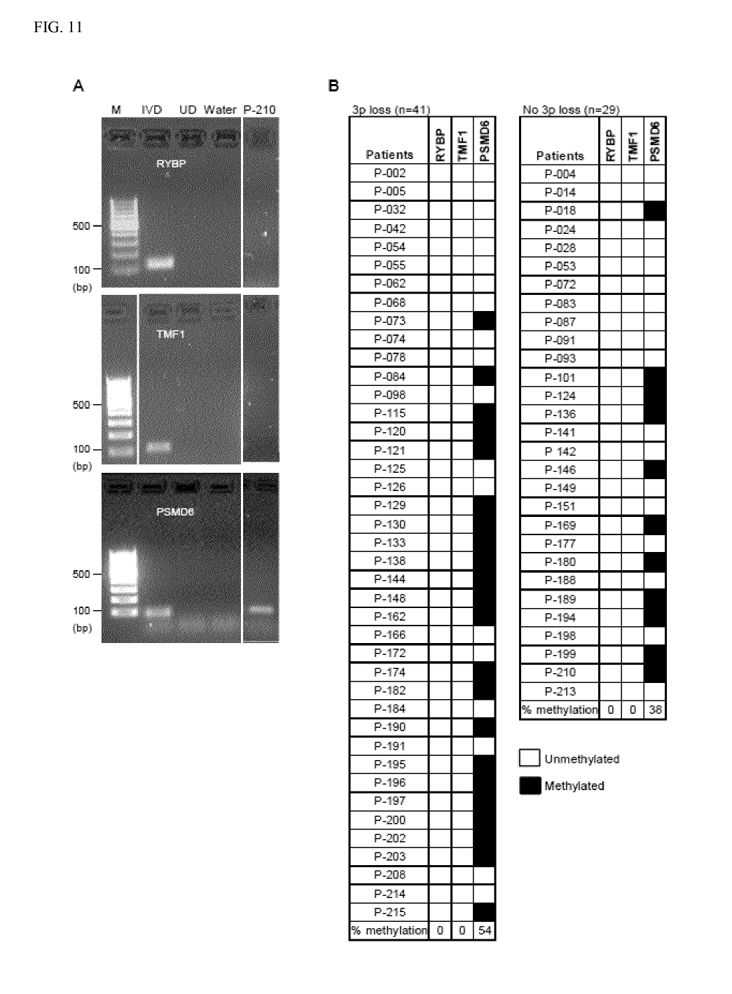

To investigate whether promoter methylation could play a role in the down-regulation of the candidate genes in addition to genetic loss, methylation analysis of three selected candidates, RYBP, TMF1, and PSMD6, was performed. For PSMD6, methylation was found in 54% (22/41) of the tumours with 3p loss and in 38% (11/29) of those without loss, including several cases with low PSMD6 expression (FIG. 11) and both tumors with 3p gain. No methylation was found for RYBP and TMF1 in any tumor at the selected promoter region.

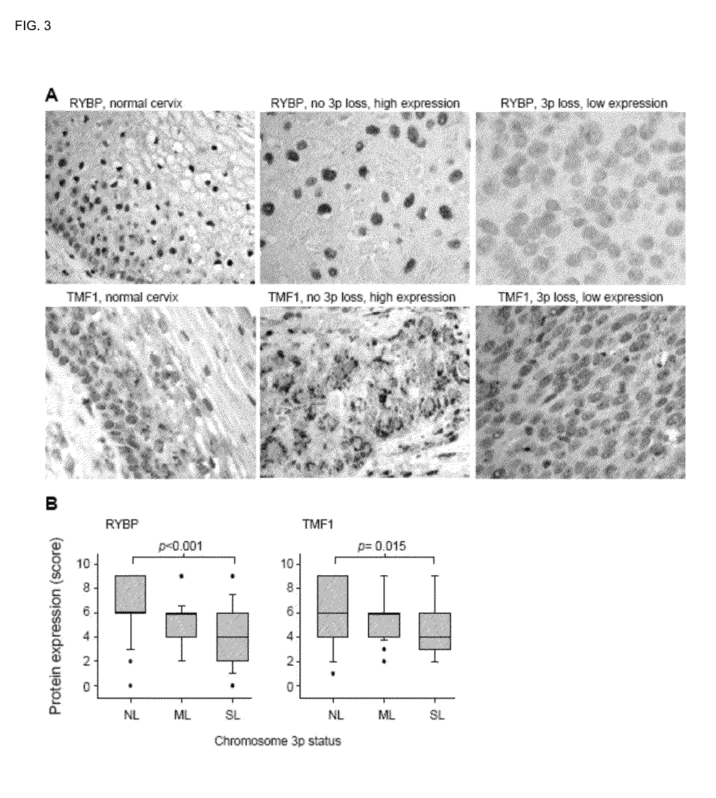

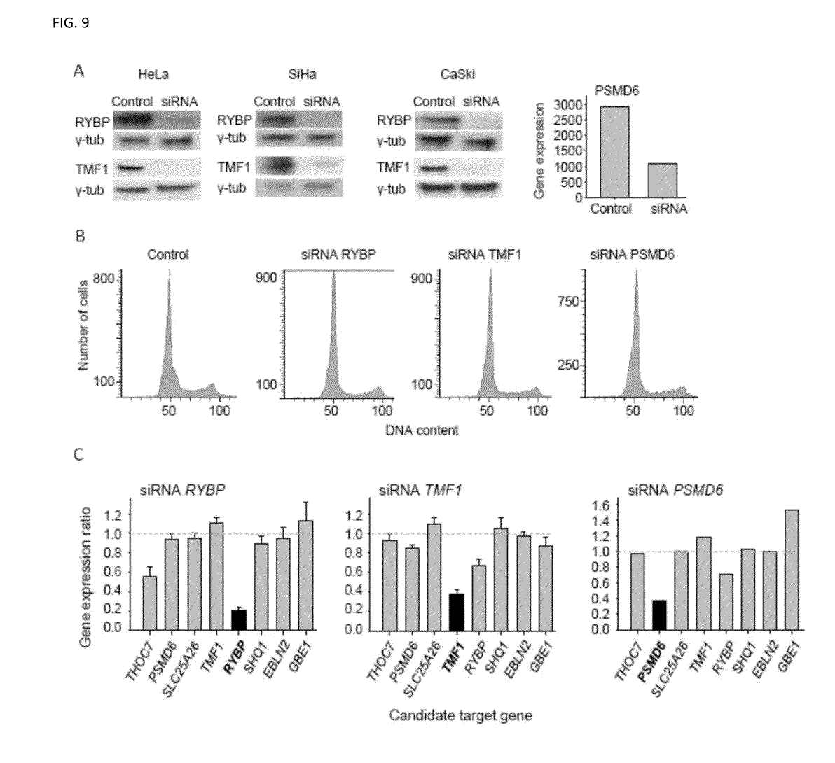

Immunohistochemistry of two selected candidates, RYBP and TMF1, was performed to validate their downregulation in tumors with 3p loss at the protein level. The protein staining indicated localization of RYBP in the nucleus and TMF1 in the Golgi (FIG. 3A, FIG. 8). RYBP and TMF1 expression showed a significant correlation to the 3p gene dosage (p<0.001 and p=0.015, respectively; FIG. 3B), in accordance with the gene expression data. Hence, RYBP protein expression was significantly downregulated in cases with moderate (p=0.003) or severe loss (p<0.001), whereas TMF1 expression was significantly downregulated in cases with severe loss (p=0.015).

Network and Biological Processes Associated with Loss of the Candidate 3p Target Genes

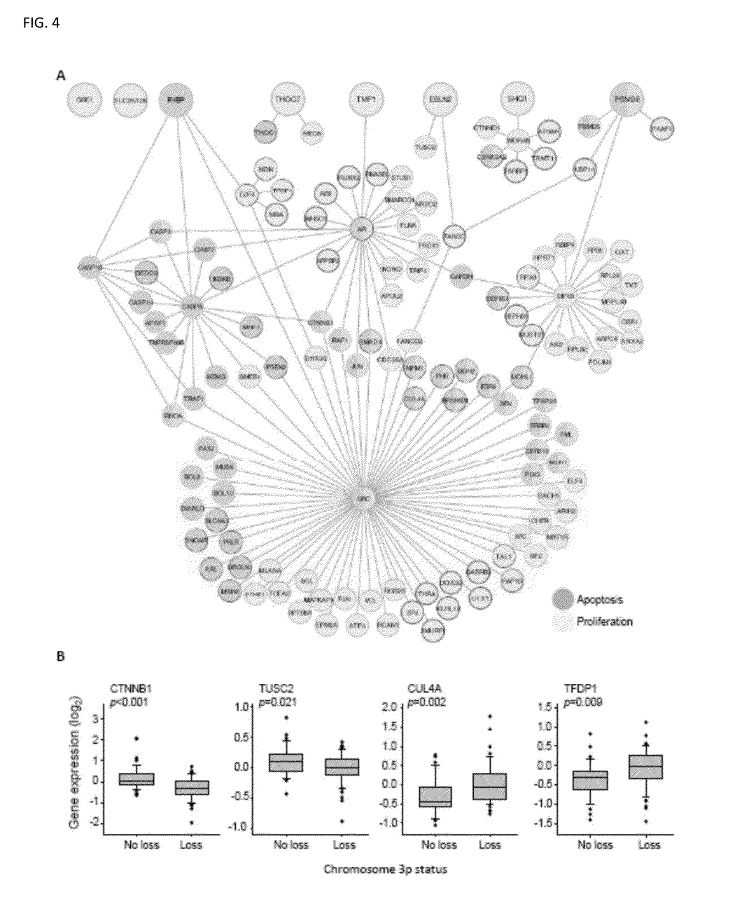

Protein interaction networks were generated around each of the eight candidates to visualize possible interaction partners that were regulated in the invasive carcinomas with loss of 3p12-p14. The second degree networks, which include only the nearest interactions, revealed a coordinate change in the expression of a number of the THOC7, SHQ1, PSMD6, TMF1, RYBP, and EBLN2 partners (SAM-GS adj p<0.0001; FIG. 4A). No network was generated for GBE1 and SLC25A26; however, this could be due to a low number of known direct interaction partners of the encoded proteins in the interaction databases (1 for GBE1, 0 for SLC25A26). Of notice was that the proposed tumor suppressors, CTNNB1 and TUSC2 (Ji et al., supra; Tian et al., supra), were among the significantly downregulated partners (p=0.001 and p=0.0005, respectively), and the significantly upregulated ones included CUL4A (p=0.005) and TFDP1 (p=0.004), which have been described as targets of the 13q34 amplification in breast cancer (Melchor et al., Breast Cancer Res 2009; 11:R86). The differential expression of these genes was confirmed in the cDNA microarray data set (FIG. 4B).

The candidate target genes PSMD6, TMF1, RYBP, and EBLN2 had several second degree interaction partners in common (FIG. 4A), and SHQ1 was connected to the nearest RYBP, TMF1, PSMD6, and EBLN2 partners through the CTNND1-CTNNB1, CSNK2A2-JUN, and CSNK2A2-SMURF1 third degree interactions, showing crosstalk in their signaling. However, this crosstalk also demonstrated that for some of the candidates, their strong downregulation in tumors with 3p loss may be caused by the loss of one of the other candidates, and not mainly by loss of the gene itself. To test this hypothesis, the gene expression changes after knockdown of three selected candidates, RYBP, TMF1, and PSMD6, were measured in three cervical cancer cell lines. Knockdown of each gene led to reduced protein expression without detectable changes in the cell cycle distribution (FIG. 9A, B). Moreover, a non-significant or small downregulation of the other candidate genes was observed (FIG. 9C), indicating that the downregulation of each of the candidate target genes was mainly caused by loss of the gene itself, at least not by loss of RYBP, TMF1, or PSMD6.

The major significant biological processes associated with the network were apoptosis, cell proliferation, cell cycle, cellular development, cellular metabolism, response to stress, and cell communication (Table 2). Visualization of each process by color coding the nodes in the network revealed connections between genes in different processes, indicating interactions between several biological processes affected by the 3p loss. This is exemplified for the apoptosis, cell proliferation, and cell cycle categories in FIG. 4A, where the two latter categories were combined in proliferation, due to their close relation.

Prognostic Impact of the Candidate 3p Target Genes

A gene signature with the expression values of the eight candidate target genes was constructed to explore the prognostic impact of all genes combined. Unsupervised hierarchical clustering divided the patients into two groups, for which the group with downregulation of the genes (cluster 2) had the highest frequency of 3p loss (83%, as compared to 23% in cluster 1; FIG. 5A) and a poor outcome compared to the other (FIG. 5B). To achieve a measure of the signature that could be compared across patients, a score was calculated a score for each tumor by averaging the median centered and log-transformed expression levels of the genes, as described previously (Chi et al., PLoS Med 2006; 3:e47). This 3p target gene score was lower for patients with 3p loss and associated with clinical outcome (FIG. 5C, D), in line with the results from the cluster analysis (FIG. 5B). The prognostic impact of the signature was further validated in an independent cohort of 74 patients, for which gene dosage data were not available (validation cohort; Table 4). Also in this cohort, a high degree of co-regulation was seen for all pairs of genes (p<0.05), except for RYBP versus GBE1 (p=0.9), RYBP versus SLC25A26 (p=0.08), and PSMD6 versus EBLN2 (p=0.08). Moreover, patients with a low 3p target gene score had a poor outcome compared to those with a high score (FIG. 5E). These results confirmed the clinical significance of the signature and supported a role of the candidate genes as targets of the 3p loss.

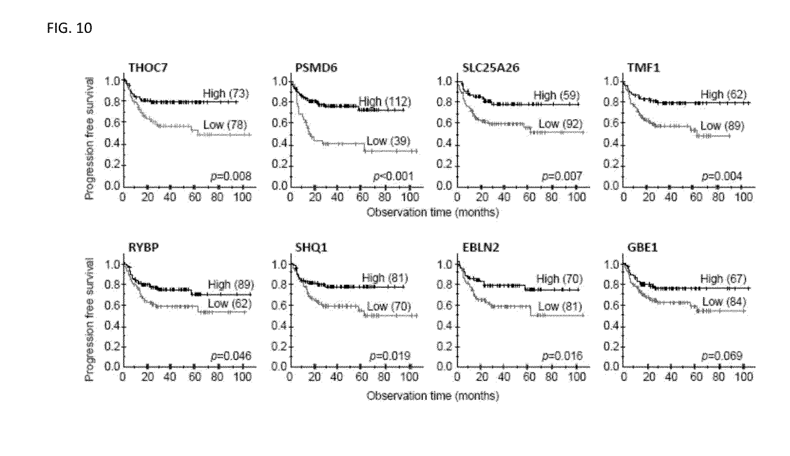

To assess the importance of the signature in comparison to existing clinical markers, the two cohorts were merged to a group of 151 patients. In univariate Cox regression analysis, the 3p target gene score was associated with both locoregional control and progression free survival (Table 3). All eight genes showed a significant or clear tendency towards a relationship to outcome in a single gene analysis (FIG. 10) and therefore seemed to contribute to the univariate result. In multivariate analysis, the score emerged as a prognostic factor independent of lymph node status, tumor size, and stage for both end points (Table 3).

TABLE 1
Candidate target genes of the 3p12-p14 loss.

| | | | 3p | Spearman correlation | | | |
|---|---|---|---|---|---|---|---|
| | Gene | | location | analysis^{b} | | | |
| Probe ID^{a} | symbol | Gene name | (Mb) | p | Adj p^{c} | R^{d} | GC-biological process |
| 5870113 | THOC7 | THO complex 7 homolog | 63.8 | 45 * 10⁻⁴ | 2.8 * 10⁻⁴ | 0.58 | Transport RNA |
| | | (Drosophila) | | | | | splicing mRNA |
| | | | | | | | processing |
| 5810070 | PSMD6 | Proteasome (prosome, macropain) | 64.0 | 4.5 * 10⁻⁷ | 2.0 * 10⁻⁴ | 0.54 | Proteolysis, apoptosis, |
| | | 26S subunit, non-ATPase, 6 | | | | | cell cycle regulation |
| 1850458 | SLC25A26 | Solute carrier family 25 member 26 | 66.5 | 5.3 * 10⁻⁴ | 0.0012 | 0.44 | Transport |
| 2970609 | TMF1 | TATA element modulatory factor 1 | 69.2 | 1.3 * 10⁻⁴ | 0.0023 | 0.42 | Transcription |
| 840554 | RYBP | RING1 and YY1 binding protein | 72.5 | 2.8 * 10⁻⁴ | 8.8 * 10⁻⁵ | 0.51 | Apoptosis, |
| | | | | | | | transcription |
| 240358 | SHQ1 | SHQ1 homolog (S. cerevisiae) | 72.9 | 1.0 * 10⁻⁶⁴ | 9.5 * 10⁻⁷ | 0.65 | RNA-protein complex |
| | | | | | | | assembly |
| 3180324 | EBLN2 | Endogenous Bornavirus-like | 73.2 | 1.7 * 10⁻⁴ | 3.1 * 10⁻⁷ | 0.62 | — |
| | | nucleoprotein 2 | | | | | |
| 6280176 | GBE1 | Glucan (1,4-alpha), branching | 81.6 | 6.2 * 10⁻⁴ | 0.010 | 0.38 | Energy metobolism |
| | | enzyme 1 | | | | | |

^{a}In the case of several probes of the same gene, the most significant probe was selected.
^{b}Analysis of gene dosage versus expression.
^{c}The p-values (p) were adjusted for multiple testing.
^{d}R. correlation coefficient.

TABLE 2
Biological processes overrepresented among
the interaction partners in the network.

| | | No. of correlating | No. of genes on | |
|---|---|---|---|---|
| GO number | GO category | genes | the array | Adj p² |
| GO: 0008150 | Biological process | 127^{b} | 13850^{b} | |
| GO: 0006915 | Apoptosis | 26 | 774 | <0.001 |
| GO: 0008283 | Cell proliferation | 21 | 765 | <0.001 |
| GO: 0007049 | Cell cycle | 18 | 796 | <0.001 |
| GO: 0048869 | Cellular development process | 35 | 1762 | <0.001 |
| GO: 0044237 | Cellular metabolic process | 103 | 7046 | <0.001 |
| GO: 0006139 | Nucleobase, nucleoside, nucleotide | 60 | 3541 | <0.001 |
| | and nucleic acid metabolic process | | | |
| GO: 0008508 | Proteolysis | 14 | 708 | 0.007 |
| GO: 0008950 | Response to stress | 22 | 1253 | 0.003 |
| GO: 0007154 | Cell communication | 50 | 3896 | 0.005 |

^{a}The p-values (p) were adjusted for multiple testing.
^{b}Genes with GO annotation (biological process).

TABLE 3
Cox regression analysis of the 3p target gene signature
and clinical variables.

| | Univariate analysis^{a} | | | Multivariate analysis^{a} | | |
|---|---|---|---|---|---|---|
| Covariate | p | HR | 95% CI | p | HR | 95% CI |

Locoregional control

| | | | | | | |
|---|---|---|---|---|---|---|
| Lymph node | 0.128 | 0.45 | 0.16-1.26 | 0.512 | — | — |
| status^{b} | | | | | | |
| FIGO stage^{c} | 0.055 | 2.71 | 0.96-7.51 | 0.195 | — | — |
| Tumor size^{d} | 0.077 | 2.64 | 0.90-7.74 | 0.040 | 3.12 | 1.05-9.20 |
| 3p target gene | 0.034 | 0.11 | 0.02-0.85 | 0.014 | 0.75 | 0.01-0.60 |
| score^{e} | | | | | | |

Progression free survival

| | | | | | | |
|---|---|---|---|---|---|---|
| Lymph node | 0.005 | 0.44 | 0.25-0.79 | 0.281 | — | — |
| status^{b} | | | | | | |
| FIGO stage^{c} | 0.00001 | 3.56 | 2.01-6.29 | 0.003 | 2.60 | 1.39-4.86 |
| Tumor size^{d} | 0.0005 | 3.18 | 1.66-6.09 | 0.003 | 2.80 | 1.42-5.51 |
| 3p target gene | 0.006 | 0.23 | 0.08-0.66 | 0.002 | 0.17 | 0.05-0.52 |
| score^{e} | | | | | | |

^{a}P-value (p), hazard ratio (HR), and 95% confidence interval (CI) are listed.
^{b}Lymph node status includes pelvic and para aortal lymph nodes.
^{c}FIGO (Federation International de Gynecologie et d'Obstetrique) stage was divided in two groups; 1b-2b and 3a-4a.
^{d}Tumor size was divided in two groups based on the median size of 43.8 cm³, corresponding to a median diameter of about 4.4 cm.
^{e}The score was calculated as the average median centered and log transformed expression of the eight genes in the 3p target signature and used as a continuous variable.

TABLE 4
Table S1. Patient and tumor charecteristics

| | All patients | aCGH cohort | Integrative cohort [symbol] | Validation cohort [symbol] |
|---|---|---|---|---|
| Characteristic | (n = 188) | (n = 92) | (n = 77) | (n = 74) |
| HPV status [symbol] | | | | |
| HPV18 | 94 | 60 | 49 | 35 |
| HPV18 | 7 | 6 | 6 | 0 |
| HPV18 + 18 | 10 | 9 | 8 | 1 |
| HPV other | 13 | 9 | 8 | 4 |
| HPV negative | 8 | 8 | 6 | 1 |
| Not determined | 33 | 0 | 0 | 33 |
| Priody [symbol] | | | | |
| 1 | 52 | 51 | 42 | 1 |
| 1.1-1.4 | 12 | 12 | 10 | 8 |
| 1.5-1.9 | 28 | 22 | 15 | 1 |
| ≧2 | 7 | 7 | 7 | 0 |
| Not determiend | 72 | 8 | 0 | 72 |
| FIGO stage (n) | | | | |
| 1B | 11 | 6 | 5 | 5 |
| 2 | 154 | 52 | 42 | 52 |
| 3 | 43 | 32 | 28 | 11 |
| 4A | 8 | 2 | 2 | 8 |
| Tumor size [symbol] [symbol] (cm³) [symbol] diameter (cm) [symbol] | | | | |
| Median | 43.6, 4.4 | 43.8, 4.4 | 48.1, 4.4 | 39.8, 4.2 |
| Range | 1.0-321, 1.8-8.8 | 2.8-321, 1.7-8.8 | 2.8-321, 1.7-8.8 | 1.0-320, 1.5-8.8 |
| Pelvic lymph node status [symbol] (n) | | | | |
| Positve | 89 | 41 | 37 | 28 |
| Negative | 97 | 51 | 40 | 48 |
| Age | | | | |
| Median | 56 | 57 | 57 | 54 |
| Range | 24-85 | 28-85 | 28-84 | 24-81 |
| Observation time (months) | | | | |
| Median | 41.1 | 80.2 | 55.7 | 26.4 |
| Range | 5.5-104 | 23-104 | 23-104 | 5.5-80.4 |
| Release (distinct and/or local) | 52 | 33 | 29 | 19 |

[symbol]

Patients with [symbol] aCGH and lumina gene expression data. [symbol]
Patients with lumina gene expression data only. [symbol]
For HPV determinations, PCR on DNA was performed, using tne primers [symbol] in [1]. The products were detected by poly [symbol] gel electropsiorsis or [symbol] Aglient DNA 1000 ul (Agilent Technologies Inc., Germany) [symbol]
Tumor priody was determined by flow [symbol] after preparation of [symbol] from a separate part of the [symbol] [ [symbol] ]. [symbol]
Tumor size and lymph nodes status were determined from [symbol] magnetic resonance [MR] images. [symbol]
Volume were calculated based on 3 orthogonal diameters (a, b, c) at [symbol] abc. [symbol]
Diameter were calculated form tumor volume [symbol] . [symbol]
indicates data missing or illegible when filed

TABLE 5
Supplementary Table S2. Primers used for methylation-
specific PCR

| | | Length of | Location | |
|---|---|---|---|---|
| | | amplicon | relative to | Tm |
| Gene^{a} | Primer sequence, 5′-3′ | (bp)^{b} | TSS (bp)^{c} | (° C.)^{d} |
| RYBP_F | GGCGTTCGGTTTTTTTTTT | 113 | ~196 to ~81 | 58 |
| | SEQ ID NO: 1 | | | |
| RYBP_R | CGAATAAACCGTCGTAATTTCG | | | |
| | SEQ ID NO: 2 | | | |
| TMF1_F | TTTGAATATTTTTTTCGGGGAAATC | 104 | ~195 to ~90 | 60 |
| | SEQ ID NO: 3 | | | |
| TMF1_R | CGAAAAAATTTCTATTAATATCGATTTCG | | | |
| | SEQ ID NO: 4 | | | |
| PSMD6_F | CGGAGACGGGATCGGAAGTC | 87 | ~98 to ~10 | 63 |
| | SEQ ID NO: 5 | | | |
| PSMD6_R | TTAATTACGACCGACTACGACAACG | | | |
| | SEQ ID NO: 6 | | | |

^{a}F: Forward primer; R: Reverse primer
^{b}bp: Base pair
^{c}TSS: Transcription start site
^{d}Tm: Melting temperature

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the field of this invention are intended to be within the scope of the following claims.

1. A kit for detecting loss of gene expression associated with cervical cancer, consisting essentially of:
a) a first gene expression informative reagent for identification of loss or decrease of gene expression of a first gene located at the chromosomal region 3p11.2-p14.2; and
b) a second gene expression informative reagent for identification of loss or decrease of gene expression of a second gene located at the chromosomal region 3p11.2-p14.2.

2. The kit of claim 1, further comprising additional gene expression informative reagents for identification of loss or decrease in gene expression in one or more additional genes.

3. The kit of claim 1, wherein said genes are selected from the group consisting of: THOC7, PSMD6, SLC25A26, TMF1, RYBP, SHQ1, EBLN2, and GBE1.

4. (canceled)

5. The kit of claim 1, wherein said first and second reagents are nucleotide probes that specifically bind to said genes.

6. The kit of claim 1, wherein said first and second reagents are antibodies that specifically bind to polypeptides encoded by said genes.

7. The kit of claim 1, wherein said first reagent is a pair of primers for amplifying said first gene and second reagent is a pair of primer for amplifying said second gene.

8. The kit of claim 1, wherein said first and second reagents are sequence primers for sequencing said first and second genes.

9. A method for detecting loss of gene expression associated with cervical cancer in a subject, comprising:
a) contacting a sample from a subject with a gene expression detection assay comprising the kit of claim 1; and
b) diagnosing said subject with cervical cancer when loss or decrease in expression of said genes is detected in said sample.

10. (canceled)

11. A method for detecting gene variants associated with cervical cancer in a subject, comprising:
a) contacting a sample from a subject with a gene expression detection assay, wherein said gene expression detection assay comprises i) a first gene expression informative reagent for identification of loss or decrease of gene expression of a first gene located at the chromosomal region 3p11.2-p14.2; and ii) a second gene expression informative reagent for identification of loss or decrease of gene expression of a second gene located at the chromosomal region 3p11.2-p14.2,
wherein said second gene is different than said first gene, under conditions that the presence of gene expression associated with cervical cancer is determined; and
b) diagnosing said subject with cervical cancer when loss or decrease in expression of said genes is detected in said sample.

12. The method of claim 11, further comprising additional gene expression informative reagents for identification of loss or decrease in gene expression in one or more additional genes.

13. The method of claim 11, wherein said genes are selected from the group consisting of: THOC7, PSMD6, SLC25A26, TMF1, RYBP, SHQ1, EBLN2, and GBE1.

14. The method of claim 11, wherein said cervical cancer is invasive cervical cancer.

15. The method of claim 11, wherein said first and second reagents are nucleotide probes that specifically bind to said genes.

16. The method of claim 11, wherein said first and second reagents are antibodies that specifically bind to polypeptides encoded by said genes.

17. The method of claim 11, wherein said first reagent is a pair of primers for amplifying said first gene and second reagent is a pair of primer for amplifying said second gene.

18. The method of claim 11, wherein said first and second reagents are sequence primers for sequencing said first and second genes.

19. The method of claim 11, wherein said sample is selected from the group consisting of a tissue sample, a cell sample, and a blood sample.

20. The method of claim 11, wherein said determining comprises detecting expression levels of nucleic acids or polypeptides from said genes.

21. The method of claim 20, wherein said detecting expression levels of nucleic acids comprises one or more nucleic acid detection method selected from the group consisting of sequencing, amplification and hybridization.

22-23. (canceled)

24. The method of claim 11, further comprising the step of treating said subject for cervical cancer.

25-27. (canceled)
